(19) United States
(12) Patent Application Publication
(10) Pub. No.: US 2026/0083834 A1
(43) Pub. Date: Mar. 26, 2026

(54) CsgA-DERIVED NANOSTRUCTURES AND USES THEREOF FOR ANTIGEN DELIVERY

(71) Applicant: TRANSFERT PLUS, LIMITED PARTNERSHIP, Quebec (CA)

(72) Inventors: Steve BOURGAULT, Montreal (CA); Denis ARCHAMBAULT, Longueuil (CA); Dominic ARPIN, Montreal (CA); Felix LAMONTAGNE, Montreal (CA)

(21) Appl. No.: 19/109,453

(22) PCT Filed: Sep. 6, 2023
(86) PCT No.: PCT/CA2023/051172

§ 371 Date: Mar. 6, 2025

Related U.S. Application Data
(60) Provisional application No. 63/374,770, filed on Sep. 7, 2022.

(51) Int. Cl. A61K 39/145; A61K 39/00; A61P 37/04; C07K 14/005; C07K 14/24

(52) U.S. Cl. CPC A61K 39/145; A61P 37/04; C07K 14/005; C07K 14/24; A61K 2039/55505; A61K 2039/6031; C12N 2760/16122; C12N 2760/16134; C12N 2760/16171

(57) ABSTRACT

Purified antigens are usually weakly immunogenic and require the addition of immunostimulatory agents and/or delivery systems to generate robust antigen-specific responses. The present application relates to self-assembling polypeptides that may be conjugated to immunogens and have the ability to self-assemble into immunogen-displaying nanofilaments that activate the humoral and cellular immune responses. The self-assembling polypeptide comprises an amino acid sequence having at least 60% identity with the sequence of the R4 and R5 domains from a Curli-specific gene A (CsgA) protein. The present application also relates to nucleic acids encoding the self-assembling polypeptide/immunogen conjugates, to compositions and vaccines comprising the self-assembling polypeptide/immunogen conjugates or nucleic acids, as well as to methods for inducing an immune response against an immunogen and/or for preventing and/or treating a microbial infection, cancer or any pathological conditions in which vaccination may be useful such as autoimmune diseases and allergies, in a subject.

### CROSS REFERENCE TO RELATED APPLICATIONS

The present application claims the benefit of U.S. provisional patent application No. 63/374,770, filed on Sep. 8, 2022, which is incorporated herein by reference.

### SEQUENCE LISTING

A sequence listing is submitted herewith as an XML file named 12810-00846-AD.xml, that was created on Aug. 24, 2023, and having a size of ˜35 kilobytes. The content of the aforementioned file is hereby incorporated by reference in its entirety.

### TECHNICAL FIELD

The present disclosure generally relates to vaccines, and more particularly to delivery vehicles and adjuvants for molecules such as immunogens.

### BACKGROUND ART

Vaccination, one of the most significant advancements for human health, has also efficiently alleviated the economic losses linked to infectious diseases in domesticated animals^{1,2}. Originally, vaccines have been composed of attenuated or inactivated, pathogens, which are respectively associated with potential safety concerns and weak immune response in young children and eldery³⁻⁵. To address these limitations, vaccine technologies that aim at inducing a robust and effective immune response while being safe have emerged. Among them, subunit vaccine formulations, which are composed of isolated microbial antigens, have shown to be effective at inducing a protective immune response combined with a high safety profile. Nonetheless, purified antigens are usually weakly immunogenic and require the addition of immunostimulatory agents and/or delivery systems to generate robust antigen-specific responses⁶⁻⁸. Among these strategies, the organized display of antigens on a nanoparticle is an efficient approach to increase the efficacy of subunit vaccines. The delivery of antigens in a (nano) particulate form enhances their stability and immunogenicity, and is associated with an increase of uptake by antigen presenting cells (APCs) and an enhanced retention at the draining lymph node, leading to a longer exposure to antigens^{7,9-15}. Moreover, particles displaying multivalent antigens can trigger the cross-linkage of B cell receptors (BCRs), which in turn enhances B cell activation and antibody production^{8,16,17}. Interestingly, owing to their size, nanoparticles diffuse passively in the lymphoid system, but do not distribute in blood capillaries, which ultimately limits potential systemic toxicity associated with smaller soluble molecules¹⁸⁻²⁰.

A large diversity of materials has been evaluated as antigen-delivery systems including inorganic particles, polymers, liposomes, and proteinaceous assemblies^{16,18}. However, the use of inorganic and polymeric particles has shown some limitations associated with potential toxicity, low biological stability, poor solubility, and/or non-biodegradability²¹. In contrast, protein-based vaccine platforms are attractive antigen carriers due to their biocompatibility, biodegradability, and the repetitive nature of assembly^{5,22,23}. The best-known protein nanovaccines currently used in the clinics and under evaluation are virus-like particles (VLPs), composed of viral structural proteins, such as vaccines against human papillomavirus (HPV) and hepatitis B virus (HBV)^{24,25}, and bacterial self-assembling protein like ferritin and lumazine synthase²⁶⁻²⁹. Nevertheless, although protein-based delivery systems are known to stabilize antigens and increase their immunogenicity, they usually have no intrinsic adjuvanticity such as activation of innate immune receptors, requiring the addition of adjuvants in vaccine formulations^{8,26}.

Thus, there is still a need to identify novel protein-based particles with intrinsic immunostimulating properties for the delivery of antigens, while minimizing potential side effects of adjuvants³⁰.

The present description refers to a number of documents, the content of which is herein incorporated by reference in their entirety.

### SUMMARY

In various aspects and embodiments, the present disclosure provides the following items 1 to 41:
1. A conjugate comprising:
   (i) a self-assembling polypeptide comprising an amino acid sequence having at least 60% identity with the sequence of the R4 and R5 domains from a Curli-specific gene A (CsgA) protein; and
   (ii) a heterologous immunogen conjugated to the self-assembling polypeptide.
2. The conjugate of item 1, wherein the self-assembling polypeptide comprises or consists of an amino acid sequence of the following formula:

(SEQ ID NO: 24)
X1-X2-X3-X4-V-X5-Q-X6-G-X7-X8-N-X9-A-X10-V-X11-Q-
X12-A-X13-X14-S-X15-V-X16-V-X17-Q-X18-G-X19-G-N-
X20-A-T-A-X21-Q-X22

wherein
   X1 is S or A, preferably S;
   X2 is E, D, T, or V; preferably E or D;
   X3 is M, I, or V, preferably M or I;
   X4 is T, N, S, V, E, or D, preferably T;
   X5 is K, G, S, R, T, or Q, preferably K or R;
   X6 is an aromatic residue, preferably F or Y;
   X7 is G, A, or T, preferably G;
   X8 is G, N, R, S, or A, preferably G;
   X9 is G, A, or D, preferably G;
   X10 is A, L, or V, preferably A;
   X11 is D or N;
   X12 is S or T, preferably T;
   X13 is S or F, preferably S;
   X14 is N, D, G, Q, or S, preferably N;
   X15 is S, T, Q, Y, N or L, preferably S;
   X16 is N, M, T, L, S, or E, preferably N;
   X17 is T, R, Q, S, H, preferably T;
   X18 is V or F, preferably V;
   X19 is F, Y or N, preferably F;
   X20 is N or H, preferably N;
   X21 is H, N, or S, preferably H; and
   X22 is Y or H, preferably Y.
3. The conjugate of item 1 or 2, wherein the self-assembling polypeptide comprises an amino acid sequence having at least 60% with any one of the sequences of SEQ ID NOs: 1 and 6 to 23.
4. The conjugate of item 3, wherein the self-assembling polypeptide comprises an amino acid sequence having at least 80% identity with any one of the sequences of SEQ ID NOs: 1 and 6 to 23.
5. The conjugate of item 3, wherein the self-assembling polypeptide comprises an amino acid sequence having at least 90% identity with any one of the sequences of SEQ ID NOs: 1 and 6 to 23.
6. The conjugate of any one of items 3 to 5, wherein the self-assembling polypeptide comprises an amino acid sequence having at least 60%, 80%, or 90% identity with the sequence of SEQ ID NO:1.
7. The conjugate of item 6, wherein the self-assembling polypeptide comprises the amino acid sequence of SEQ ID NO:1.
8. The conjugate of any one of items 1 to 7, wherein the self-assembling polypeptide has a length of 60, 50, or 45 amino acids or less.
9. The conjugate of any one of items 1 to 8, wherein the immunogen is a protein from a microorganism or a peptide fragment thereof comprising at least 10 amino acids.
10. The conjugate of item 9, wherein the immunogen is a viral protein, a bacterial protein, a fungal protein, a protozoan protein, a helminth protein, or a peptide fragment thereof.
11. The conjugate of item 10, wherein the is a viral protein or a peptide fragment thereof.
12. The conjugate of item 11, wherein the viral protein or peptide fragment thereof is a protein from influenza virus or a peptide fragment thereof.
13. The conjugate of item 12, wherein the immunogen is a peptide fragment derived from the extracellular domain of the influenza M2 protein (M2e).
14. The conjugate of any one of items 1 to 8, wherein the immunogen is a tumor-specific antigen.
15. The construct of any one of items 1 to 14, wherein the immunogen is a peptide fragment of 10 to 50 amino acids.
16. The conjugate of item 15, wherein the immunogen comprises the sequence SLLTEVETPIRNEWGSRSNGSSD (SEQ ID NO:2).
17. The conjugate of item 16, wherein the immunogen comprises three repeats of the sequence of SEQ ID NO:2.
18. The conjugate of item 17, wherein the immunogen comprises the sequence of SEQ ID NO: 27.
19. The conjugate of any one of items 1 to 18, further comprising a linker between the immunogen and the self-assembling polypeptide.
20. The conjugate of item 19, wherein the linker is a peptide linker of 4 to 16 amino acids.
21 The conjugate of item 20, wherein the linker is a peptide linker of 4 to 10 amino acids.
22. The conjugate of any one of items 19 to 21, wherein the linker comprises glycine and/or serine residues.
23. The conjugate of item 22, wherein the linker comprises the motif (GGGS)ₙ (SEQ ID NO: 26), wherein n is an integer from 1 to 4.
24. The conjugate of item 23, wherein n is 2.
25. The conjugate of any one of items 1 to 24, wherein the immunogen is conjugated to the amino-terminal end of the self-assembling polypeptide.
26. The conjugate of any one of items 1 to 25, wherein the conjugate assembled into nanofilaments having a diameter of about 2 to about 20 nm, or of about 5 to about 10 nm.
27. A nucleic acid encoding the conjugate of any one of items 1 to 26.
28. A composition comprising the conjugate of any one of items 1 to 26 or the nucleic acid of item 27.
29. The composition of item 28, further comprising a pharmaceutically acceptable carrier.
30. A vaccine comprising the conjugate of any one of items 1 to 26, the nucleic acid of item 27, or the composition of item 28 or 29.
31. The vaccine of item 30, wherein the vaccine further comprises an adjuvant.
32. A method for inducing an immune response against an immunogen in a subject comprising administering to the subject an effective amount of: (i) the conjugate of any one of items 1 to 26; (ii) the nucleic acid of item 27; (iii) the composition of item 28 or 29; or (iv) the vaccine of item 30 or 31.
33. A method for preventing and/or treating a microbial infection, an autoimmune disease, an allergy or cancer in a subject comprising administering to the subject an effective amount of: (i) the conjugate of any one of items 1 to 26; (ii) the nucleic acid of item 27; (iii) the composition of item 28 or 29; or (iv) the vaccine of item 30 or 31.
34. Use of (i) the conjugate of any one of items 1 to 26; (ii) the nucleic acid of item 27; (iii) the composition of item 28 or 29; or (iv) the vaccine of item 30 or 31, for inducing an immune response against an immunogen in a subject.
35. Use of (i) the conjugate of any one of items 1 to 26; (ii) the nucleic acid of item 27; (iii) the composition of item 28 or 29; or (iv) the vaccine of item 30 or 31, for the manufacture of a medicament for inducing an immune response against an immunogen in a subject.
36. Use of (i) the conjugate of any one of items 1 to 25; (ii) the nucleic acid of item 26; (iii) the composition of item 27 or 28; or (iv) the vaccine of item 29, for preventing and/or treating a microbial infection, an autoimmune disease, an allergy or cancer in a subject.
37. Use of (i) the conjugate of any one of items 1 to 26; (ii) the nucleic acid of item 27; (iii) the composition of item 28 or 29; or (iv) the vaccine of item 30 or 31, for the manufacture of a medicament for preventing and/or treating a microbial infection, an autoimmune disease, an allergy or cancer in a subject.
38. An agent for use in inducing an immune response against an immunogen in a subject, wherein the agent is (i) the conjugate of any one of items 1 to 26; (ii) the nucleic acid of item 27; (iii) the composition of item 28 or 29; or (iv) the vaccine of item 30 or 31.
39. An agent for use in preventing and/or treating a microbial infection, an autoimmune disease, an allergy or cancer in a subject, wherein the agent is (i) the conjugate of any one of items 1 to 26; (ii) the nucleic acid of item 27; (iii) the composition of item 28 or 29; or (iv) the vaccine of item 30 or 31.
40. The (i) the conjugate of any one of items 1 to 26; (ii) the nucleic acid of item 27; (iii) the composition of item 28 or 29; or (iv) the vaccine of item 30 or 31, for use as a medicament.
41. A method for improving or increasing the immunogenicity of an immunogen comprising conjugating the immunogen to the self-assembling polypeptide defined in any one of items 1 to 26.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts a Coomassie Blue stained SDS-PAGE analysis of purified 3M2e-R4R5 and 3M2e-CsgA proteins.

FIG. 2 depicts a Coomassie Blue stained SDS-PAGE analysis of purified eGFP-R4R5.

FIGS. 3A and 3B depict the sequences and schematic representations of full length CsgA protein (FIG. 3A) and engineered R4R5-CsgA polypeptide (FIG. 3B) in fusion with three repetitions of the M2e antigenic sequence from the influenza A virus. 3M2e=SEQ ID NO: 27; Linker=SEQ ID NO: 25; CsgA R1 domain=SEQ ID NO: 3; CsgA R2 domain=SEQ ID NO: 4; CsgA R3 domain=SEQ ID NO: 5; CsgA R4 domain=SEQ ID NO: 28; CsgA R5 domain=SEQ ID NO: 29.

FIGS. 4A-4H show the characterization of self-assembly of CsgA protein or R4R5 domain displaying the 3M2e polypeptide into cross-β-sheet filaments. Proteins were left to self-assemble in PBS for 24 h at room temperature (RT) at a concentration of 600 μg/mL under fully quiescent conditions. FIGS. 4A, B: Negative stain electron microscopy of assembled (24 h) 3M2e-CsgA (FIG. 4A) and 3M2e-R4R5 (FIG. 4B). Scale bar is 100 nm. FIG. 4C: Antigen accessibility on CsgA-based assemblies by indirect ELISA with anti-M2e antibody. FIG. 4D: Far-UV CD spectra of freshly purified (0 h) and assembled (24 h) 3M2e-R4R5 and 3M2e-CsgA proteins. FIGS. 4E, F: Fluorescence spectra of ThT (FIG. 4E) and ANS (FIG. 4F) after excitation at 440 nm and 370 nm, respectively of CsgA-based chimeric proteins. FIGS. 4G, H: Viscosity (FIG. 4G) and turbidity (FIG. 4H) analysis of freshly purified (0 h) and assembled (24 h) 3M2e-CsgA and 3M2e-R4R5 solutions. Data shows the means±standard deviation (S.D.) of three separate experiments. Statistical significance was established using a student's t-test with *P<0.05, ****P<0.0001.

FIG. 5A shows representative transmission electron microscope (TEM) negatively stained images of aggregates formed by the assembly of 3M2e-CsgA (left image) and 3M2e-R4R5 (right image). Proteins were assembled in PBS for 24 h at RT at a concentration of 600 μg/mL under fully quiescent conditions.

FIG. 5B shows representative AFM images of aggregates formed by the assembly of 3M2e-CsgA (left image) and 3M2e-R4R5 (right image). Proteins were assembled in PBS for 24 h at RT at a concentration of 600 μg/mL under fully quiescent conditions.

FIG. 6 is an image showing the assessment of solution viscosity by tube inversion of assembled 3M2e-CsgA (left tube) and 3M2e-R4R5 (right tube). Proteins were assembled in PBS for 24 h at RT at a concentration of 600 μg/mL under fully quiescent conditions.

FIGS. 7A-D show that CsgA-based assemblies activate TLR2 and induce IL-1β secretion independently of cell-death. FIG. 7A: TLR2-TLR1 stimulation by CsgA assemblies. HEK-Blue cells expressing the heterodimer TLR2-TLR1 were exposed to increasing concentrations of CsgA filaments for 16 h and activation was measured using SEAP reporter under NF-κB/AP-1 promoter. FIG. 7B: IL-1β secretion by macrophages. J774.A1 cells were incubated for 16 h with CsgA filaments and level of IL-1β in the supernatant was measured by ELISA. FIGS. 7C, 7D: Viability of macrophages upon treatment with CsgA. FIG. 7C: J774.A1 cells were treated with CsgA filaments for 16 h and metabolic activity was measured by means of resazurin reduction. FIG. 7D: Representative fluorescence microscopy images showing the distribution of live (middle images) and dead (left images) J774.A1 cells after treatment with 30 μg/mL of 3M2e-CsgA and 3M2e-R4R5 for 16 h. Positive control cells were treated with 70% methanol for 10 minutes before staining. Scale bar: 100 μm. For FIGS. 7A-7C, data represents the mean±S.D. of at least three experiments performed in triplicate.

FIGS. 8A-8F show the internalization of CsgA nanofilaments by APCs and maturation of dendritic cells. FIG. 8A: TEM image of eGFP-R4R5 after 24 h incubation in PBS at a concentration of 600 μg/mL under quiescent conditions. Internalization by dendritic cells (DC2.4) (FIGS. 8B, 8D) and macrophages (J774.A1) (FIGS. 8C, 8E) analyzed by flow cytometry (FIGS. 8B, 8C) and confocal (FIGS. 8D, 8E). Cells were treated with 5 μg/mL (FIGS. 8B, 8C) and 30 μg/mL (FIGS. 8D, 8E) of eGFP-R4R5 or soluble eGFP for 3 h with followed by extensive washing to remove membrane-bound fibrils. For flow cytometry, cells were quenched in 50% (v/v) trypan blue, right before analysis to remove surface fluorescence. For FIGS. 8B and 8C, data represents the mean±S.D. of three experiments done in triplicate. Statistical significance was analyzed with a two-way ANOVA with Tukey's multiple comparisons test (****P<0.0001). Left bars=eGFP-R4R5; right bars=eGFP. FIG. 8F: Flow cytometry analysis of dendritic cells (DC2.4) maturation following treatment with CsgA-based assemblies for 16 h. Fold-expression is determined according to untreated cells. Data represents the mean±S.D. of three experiments in triplicate. Statistical significance was analyzed with a one-way ANOVA with Tukey's multiple comparisons test (*P<0.05; ***P<0.001; ****P<0.0001).

FIGS. 9A-9F show that intramuscular immunization with CsgA-based nanofilaments induces strong anti-M2e antibody response and protects mice against experimental infection with influenza A virus. FIG. 9A: Immunization, sera collection, and experimental challenge timeline. Mice were immunized intramuscularly with 18 μg of 3M2e (in PBS or in 50% [v/v] Alum), 30 μg of 3M2e-R4R5, or 50 μg of 3M2e-CsgA. FIG. 9B: Weight loss curve post-1^{st} immunization dose. Data represents mean±standard error of the mean (S.E.M.) and statistical significance between groups was established using one-way ANOVA with Tukey's multiple comparisons test (**P<0.01; ***P<0.001). FIG. 9C: Total anti-M2e IgG in mice sera after 1^{st}, 2^{nd}, and 3^{rd} immunization(s). Mice were immunized intramuscularly with 18 μg of 3M2e (in PBS or in 50% [v/v] Alum), 30 μg of 3M2e-R4R5, or 50 μg of 3M2e-CsgA. Data represents mean and individual values of mice (n=12 per group) and statistical significance between groups was established using one-way ANOVA with Tukey's multiple comparisons test (**P<0.01; ***P<0.001 ****P<0.0001). FIGS. 9C-9F: Two weeks after the 3^{rd} immunization dose, mice were inoculated intranasally with 5× median lethal dose (LD₅₀) of influenza A virus (IAV) H1N1 PR8. FIG. 9D: Survival percentage curve of infected mice in each group (n=8). FIG. 9E, 9F: Mice were monitored daily to evaluate weight loss (FIG. 9E) and clinical score (FIG. 9F). For FIGS. 9D-9F, data represents mean±S.E.M. and for FIG. 9D statistical significance was obtained following a log-rank Mentel-Cox test. (***P<0.001).

FIGS. 10A-10C show that 3M2e-expressing nanofilaments induce a mixed Th1/Th2 M2e-specific immune response in mice. FIG. 10A: M2e-specific IgG isotypes in mice sera following the 3^{rd} immunization. Sera were diluted (IgG1: 1/16 000; IgG2a and IgG2b: 1/1000; IgG3: 1/65). FIG. 10B: IFNγ and IL-4 ELISPOT analysis of ex vivo stimulated splenocytes. Splenocytes were stimulated for 36 h with 2 μg of M2e peptide. For FIGS. 10A and 10B, data represents mean and individual value of n=4 mice per group and statistical significance was obtained following one-way ANOVA analysis with Tukey's multiple comparison (*P<0.05; **P<0.01; ***P<0.001; ****P<0.0001). FIG. 10C: ELISA analysis of interferon gamma (IFNγ) and interleukin-4 (IL-4) production by M2e-stimulated splenocytes for 72 h (n=4 mice per group). Data represents mean±S.E.M. and statistical significance was obtained following one-way ANOVA analysis with Tukey's multiple comparisons test (*P<0.05; **P<0.01; ***P<0.001; ****P<0.0001).

FIGS. 11A-11D show that CsgA-based nanofilaments do not induce overt inflammation. FIGS. 11A, 11B: Serum IL-6 (FIG. 11A) and TNFα (FIG. 11B) levels at 2, 6, and 24 h post intraperitoneal (IP) administration with 20 μM of CsgA-based nanofilaments, soluble 3M2e, or FljB-3M2e (n=6 per group). FIGS. 11C, 11D: Percentage of initial weight (FIG. 11C) and temperature (FIG. 11C) at 2, 6, and 24 h after IP immunization (n=5 per group). Data represents the mean±S.D. and statistical significance was obtained following a two-way ANOVA with Tukey's multiple comparison test ((**P<0.01; ***P<0.001; ****P<0.0001).

FIGS. 12A-12D show that intranasal immunization with 3M2e-R4R5-based nanofilaments reduces clinical score and protects mice against experimental infection with influenza A virus. FIG. 12A: Schedule of immunization and influenza experimental challenge. Weight loss (FIG. 12B), clinical score (FIG. 12C) and survival (FIG. 12D) of mice immunized with 3M2e-R4R5, 3M2e, or vehicle (PBS) following infection with 5 LD₅₀ of influenza virus, strain A/Puerto Rico/8/1934. For survival, log rank Mantel-Cox statistical test was used.

FIGS. 13A-H show that intranasal immunization with 3M2e-R4R5-based nanofilaments induces strong anti-M2e antibody response. Total IgG titers from mice sera after one immunization (FIG. 13A) or two immunizations (FIG. 13B). Secretory IgA (SIgA) titers in bronchoalveolar lavage fluid (BALF, FIG. 13C) or nasal-associated lymphoid tissue (NALT, FIG. 13D). FIGS. 13E-13H: Sera IgG isotypes OD from indirect ELISA. For IgG1 (FIG. 13E) and IgG2a (FIG. 13F), sera dilution shown is 1:8320 and 1:2080, respectively, and for IgG2b (FIG. 13G) and IgG3 (FIG. 13H), sera dilution shown is 1:1040. For titers and IgG isotypes, one way ANOVA followed by Tukey's multiple comparison test was used. Student's t-test was used for NALT sIgA. *p>0.05, **p>0.01 ***p>0.001.

FIGS. 14A-F show intranasal immunization with 3M2e-R4R5-based nanofilaments induces a cellular immune response. IFN-γ was quantified from splenocytes cultured with M2e peptide by ELISpot (FIG. 14A) and ELISA (FIG. 14B), and from lungs cells by ELISA (FIG. 14C). FIGS. 14D-E: Alveolar macrophages were exposed to LPS and the pro-inflammatory cytokines tumor necrosis factor-alpha (TNF-α, FIG. 14D) and interleukin-6 (IL-6, FIG. 14E) were quantified by ELISA. FIG. 14F: Interleukin-17 (IL-17) from lung cells cultured with M2e peptide quantified by ELISA. Student's t-test was used. *p>0.05, **p>0.01 ***p>0.001.

FIGS. 15A-D show the cytokines profile of memory T cells following intranasal immunization with 3M2e-R4R5-based nanofilaments. Splenocytes and lung cells were cultured with M2e peptide and IFN-γ, IL-2 and TNF-α from memory T cells was measured by flow cytometry. FIGS. 15A-15B: Cytokines producing CD4+ T effector memory cells (CD4+ TEM, CD44+, CD62L−) from lungs (FIG. 15A) and spleen (FIG. 15B). FIG. 15C: Cytokines producing CD4+ T central memory cells (CD4+ TCM, CD44+, CD62L+) from splenocytes. FIG. 15D: Cytokines producing CD8+ T central memory cells (CD8+ TCM) from splenocytes.

FIG. 16 shows an amino acid sequence alignment of the CsgA R4R5 domains from various bacterial species and strains, with the amino acid variations relative to SEQ ID NO:1 underlined. SEQ ID NO:1=CsgA R4R5 domains from various Escherichia coli strains including K12, SEQ ID NO: 6=CsgA R4R5 domains from E. coli strain O6:H1 (SEQ ID NO:6), SEQ ID NO:7=CsgA R4R5 domains from several Salmonella enterica species including serovars Typhimurium and Enteritidis, SEQ ID NO:8=CsgA R4R5 domains from Enterobacter ludwigii (SEQ ID NO:8), SEQ ID NO: 9=CsgA R4R5 domains from Citrobacter rodentium strain ICC168 (SEQ ID NO:9), SEQ ID NO: 10=CsgA R4R5 domains from Pseudomonas tritici, SEQ ID NO: 11=CsgA R4R5 domains from Enterobacteriaceae bacterium strain ENNIH1, SEQ ID NO:12=CsgA R4R5 domains from Citrobacter koseri strain ATCC BAA-895/CDC 4225-83/SGSC4696, SEQ ID NO:13=CsgA R4R5 domains from Trabulsiella guamensis strain ATCC 49490, SEQ ID NO: 14=CsgA R4R5 domains from Pseudescherichia vulneris strain NBRC 102420, SEQ ID NO: 15=CsgA R4R5 domains from Citrobacter tructae, SEQ ID NO:16=CsgA R4R5 domains from Pseudescherichia vulneris strain NBRC 102420, SEQ ID NO:17=CsgA R4R5 domains from Beauveria bassiana D1-5, SEQ ID NOs: 18 and 19=CsgA R4R5 domains from strains of Shigella flexneri, SEQ ID NO:20=CsgA R4R5 domains from Klebsiella pneumoniae, SEQ ID NO:21=CsgA R4R5 domains from Shigella dysenteriae, SEQ ID NO:22=CsgA R4R5 domains from Lelliottia amnigena, and SEQ ID NO:23=CsgA R4R5 domains from Pseudenterobacter timonensis. The percentage of sequence identity with SEQ ID NO:1 are indicated between parentheses after the sequence identifiers.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device, or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have shown that an antigen delivery system comprising the R4 and R5 domains of the protein Curli-specific gene A (CsgA) fused to three repeats of the M2e antigenic sequence from the influenza A virus spontaneously self-assembled into antigen-displaying nanofilaments that activate the heterodimeric toll-like receptors 2 and 1 (TLR2-TLR1). The nanofilaments were shown to be internalized by antigen presenting cells, to stimulate the maturation of dendritic cells, and to induce robust and protective humoral and cellular immune responses against H1N1 influenza A virus without inducing signs of systemic inflammation and reactogenicity after mice inoculation.

The present disclosure provides a conjugate comprising (i) a self-assembling polypeptide comprising or consisting of an amino acid sequence having at least 60% identity with the sequence of the R4 and R5 domains from a Curli-specific gene A (CsgA) protein; and (ii) a heterologous immunogen conjugated to the self-assembling polypeptide.

CsgA a bacterial protein expressed by numerous enteric bacteria and that is the main extracellular matrix component contributing to biofilm formation^{32,33}. CsgA is composed of five imperfect repeating domains or units (R1-R5) that fold into a β-sheet-turn-β-sheet secondary conformational motif that are stacked on top of one another, and the β-helix supramolecular structure is stabilized by intermolecular hydrogen bond ladders involving asparagine and glutamine residues^{36,37}. The R4 and R5 domains are the two domains located at the C-terminal end of CsgA (i.e., the last 40-45 amino acids). As shown in the studies described herein, a polypeptide comprising the R4R5 domains of CsgA has the ability to self-assemble into β-sheet-rich secondary structures (with limited or no protein aggregation), which confer it with potent immunostimulating properties. Accordingly, it is expected that any polypeptide with a structure similar to the R4 and R5 units of CsgA protein and having the ability to self-assemble into β-sheet-rich secondary structures would be suitable for incorporation into the conjugate described herein.

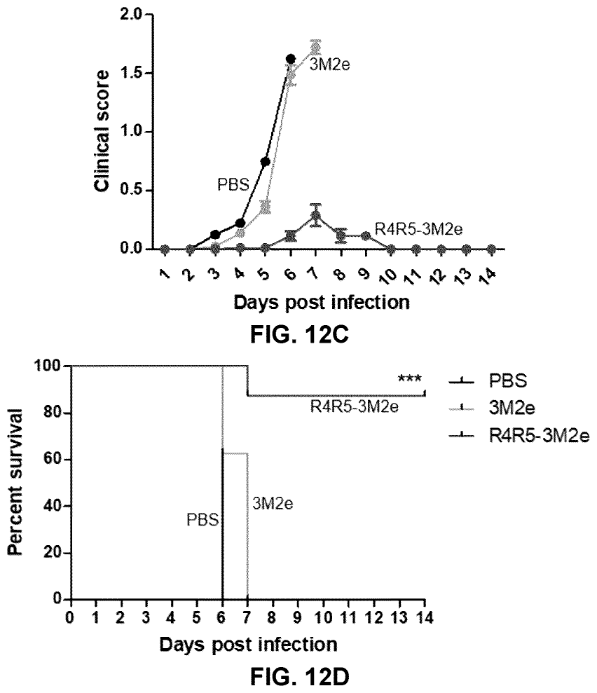

The amino acid sequences of representative R4 and R5 domains of CsgA from several bacterial strains/species are depicted in FIG. 12. SEQ ID NO:1=CsgA R4R5 domains from various Escherichia coli strains including K12, SEQ ID NO:6=CsgA R4R5 domains from E. coli strain 06: H1 (SEQ ID NO:6), SEQ ID NO:7=CsgA R4R5 domains from several Salmonella enterica species including serovars Typhimurium and Enteritidis, SEQ ID NO:8=CsgA R4R5 domains from Enterobacter ludwigii (SEQ ID NO:8), SEQ ID NO:9=CsgA R4R5 domains from Citrobacter rodentium strain ICC168 (SEQ ID NO:9), SEQ ID NO: 10=CsgA R4R5 domains from Pseudomonas tritici, SEQ ID NO:11=CsgA R4R5 domains from Enterobacteriaceae bacterium strain ENNIH1, SEQ ID NO:12=CsgA R4R5 domains from Citrobacter koseri strain ATCC BAA-895/CDC 4225-83/SGSC4696, SEQ ID NO:13=CsgA R4R5 domains from Trabulsiella guamensis strain ATCC 49490, SEQ ID NO:14=CsgA R4R5 domains from Pseudescherichia vulneris strain NBRC 102420, SEQ ID NO:15=CsgA R4R5 domains from Citrobacter tructae, SEQ ID NO:16=CsgA R4R5 domains from Pseudescherichia vulneris strain NBRC 102420, SEQ ID NO:17=CsgA R4R5 domains from Beauveria bassiana D1-5, SEQ ID NOs: 18 and 19=CsgA R4R5 domains from strains of Shigella flexneri, SEQ ID NO:20=CsgA R4R5 domains from Klebsiella pneumoniae, SEQ ID NO:21=CsgA R4R5 domains from Shigella dysenteriae, SEQ ID NO: 22=CsgA R4R5 domains from Lelliottia amnigena, and SEQ ID NO:23=CsgA R4R5 domains from Pseudenterobacter timonensis.

In an embodiment, the self-assembling polypeptide comprises or consists of an amino acid sequence of the following formula:

(SEQ ID NO: 24)
X1-X2-X3-X4-V-X5-Q-X6-G-X7-X8-N-X9-A-X10-V-X11-Q-
X12-A-X13-X14-S-X15-V-X16-V-X17-Q-X18-G-X19-G-N-
X20-A-T-A-X21-Q-X22

wherein
X1 is S or A, preferably S;
X2 is E, D, T, or V; preferably E or D;
X3 is M, I, or V, preferably M or I;
X4 is T, N, S, V, E, or D, preferably T;
X5 is K, G, S, R, T, or Q, preferably K or R;
X6 is an aromatic residue, preferably F or Y;
X7 is G, A, or T, preferably G;
X8 is G, N, R, S, or A, preferably G;
X9 is G, A, or D, preferably G;
X10 is A, L, or V, preferably A;
X11 is D or N;
X12 is S or T, preferably T;
X13 is S or F, preferably S;
X14 is N, D, G, Q, or S, preferably N;
X15 is S, T, Q, Y, N or L, preferably S;
X16 is N, M, T, L, S, or E, preferably N;
X17 is T, R, Q, S, H, preferably T;
X18 is V or F, preferably V;
X19 is F, Y or N, preferably F;
X20 is N or H, preferably N;
X21 is H, N, or S, preferably H; and
X22 is Y or H, preferably Y.

In embodiments, the self-assembling polypeptide comprises or consists of an amino acid sequence having at least 65%, 70%, 75%, 80%, 85%, 90%, 95%, 96%, 97%, 98%, or 99% identity with the sequence of any one of SEQ ID NOs: 1 and 6 to 23. In a further embodiment, the self-assembling polypeptide of the present disclosure comprises or consists of the sequence of any one of SEQ ID NOs: 1 and 6 to 23.

In embodiments, the self-assembling polypeptide comprises or consists of an amino acid sequence having at least 60%, 65%, 70%, 75%, 80%, 85%, 90%, 95%, 96%, 97%, 98%, or 99% identity with the sequence of SEQ ID NO:1.

The term “self-assembling polypeptide” as used herein refers to polypeptides whose chemical properties are such that they spontaneously form supramolecular structures in vitro or in vivo. The self-assembling polypeptide of the present disclosure has the ability to spontaneously form β-helix supramolecular structure (nanofilaments). In an embodiment, the nanofilaments have a diameter of about 2 to about 20 nm, or about 4 to about 15 nm, or about 5 to about 10 nm. In an embodiment, the nanofilaments have a length of about 500 nm to about 10 μm, or about 1 μm to about 3 μm.

In an embodiment, the self-assembling polypeptide of the present disclosure does not comprise the full-length sequence of a CsgA protein. In an embodiment, the self-assembling polypeptide of the present disclosure lacks full R1 the (e.g., sequence SELNIYQYGGGNSALALQTDARN, SEQ ID NO:3, for E. coli strain K12), R2 (e.g., sequence SDLTITQHGGGNGADVGQGSDD, SEQ ID NO:4, for E. coli strain K12), and/or R3 (e.g., sequence SSIDLTQRGFGNSATLDOWNGKN, SEQ ID NO:5, for E. coli strain K12) domains of a CsgA protein. In an embodiment, the self-assembling polypeptide of the present disclosure lacks at least two of the full R1, R2, and/or R3 domains of a CsgA protein. In a further embodiment, the self-assembling polypeptide of the present disclosure lacks the full R1, R2, and R3 domains of a CsgA protein.

The self-assembling polypeptide may comprise L- and D-isomers of the naturally occurring amino acids, as well as other amino acids (e.g., naturally-occurring amino acids, non-naturally-occurring amino acids, amino acids which are not encoded by nucleic acid sequences, etc.) used in peptide chemistry to prepare synthetic analogs of peptides. Examples of naturally-occurring amino acids are glycine, alanine, valine, leucine, isoleucine, serine, threonine, etc. Other amino acids include for example non-genetically encoded forms of amino acids, as well as a conservative substitution of an L-amino acid. Naturally-occurring non-genetically encoded amino acids include, for example, beta-alanine, 3-amino-propionic acid, 2,3-diamino propionic acid, alpha-aminoisobutyric acid (Aib), 4-aminobutyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, or 2,3-diaminobutyric acid (D- or L-), etc. These amino acids are well known in the art of biochemistry/peptide chemistry.

The above-noted self-assembling polypeptide may comprise all L-amino acids, all D-amino acids, or a mixture of L- and D-amino acids. As such, the single-letter code for designing amino acids in the above-noted formula encompass both the L- and D-isomers of the recited amino acids (for those having a chiral center). For example, the letter “N” refers to L-asparagine and D-asparagine. In an embodiment, the self-assembling polypeptide comprises only L-amino acids.

“Identity” refers to sequence similarity/identity between two polypeptide molecules. The identity can be determined by comparing each position in the aligned sequences. A degree of identity between amino acid sequences is a function of the number of identical amino acids at positions shared by the sequences. As used herein, a given percentage of identity between sequences denotes the degree of sequence identity in optimally aligned sequences.

Optimal alignment of sequences for comparisons of identity may be conducted using a variety of algorithms, such as the local homology algorithm of Smith and Waterman, 1981, Adv. Appl. Math 2:482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerized implementations of these algorithms (such as GAP, BESTFIT, FASTA and TFASTA in the Wisconsin Genetics Software Package, Genetics Computer Group, Madison, WI, U.S.A.). Sequence similarity or identity may also be determined using the BLAST algorithm, described in Altschul et al., 1990, J. Mol. Biol. 215:403-10 (using the published default settings). Software for performing BLAST analysis may be available through the National Center for Biotechnology Information web site. The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighborhood word score threshold. Initial neighborhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89:10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P[N] which provides an indication of the probability by which a match between two nucleotide or amino acid sequences would occur by chance.

“Variant” as used herein refers to a self-assembling polypeptide in which one or more of the amino acids of the native sequence has/have been modified, but which retains adjuvant, immunostimulatory, and/or immunopotentiating activity. The modification may be, for example, a deletion of one or more consecutive or non-consecutive amino acids, a substitution of amino acids, one or more substitution(s) of a naturally occurring amino acid (L-amino acid) by a corresponding D-amino acid, an extension of the sequence by e.g., one, two, three or more amino acids. In an embodiment, the above-mentioned substitution(s) are conserved amino acid substitutions. As used herein, the term “conserved amino acid substitutions” (or sometimes “conservative amino acid substitutions”) refers to the substitution of one amino acid for another at a given location in the self-assembling polypeptide, where the substitution can be made without substantial loss of the relevant structure/function (e.g., ability to self-assemble). In making such changes, substitutions of like amino acid residues can be made on the basis of relative similarity of side-chain substituents, for example, their size, charge, hydrophobicity, hydrophilicity, and the like, and such substitutions may be assayed for their effect on the structure/function of the self-assembling polypeptide by routine testing.

In some embodiments, conserved amino acid substitutions may be made where an amino acid residue is substituted for another having a similar hydrophilicity value (e.g., within a value of plus or minus 2.0), where the following may be an amino acid having a hydropathic index of about −1.6 such as Tyr (−1.3) or Pro (−1.6) are assigned to amino acid residues (as detailed in U.S. Pat. No. 4,554,101): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4).

In other embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gln (−3.5); Asp (−3.5); Asn (−3.5); Lys (−3.9); and Arg (−4.5).

In other embodiments, conserved amino acid substitutions may be made where an amino acid residue is substituted for another in the same class, where the amino acids are divided into non-polar, acidic, basic and neutral classes, as follows: non-polar: Ala, Val, Leu, Ile, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-aminobutyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In other embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (J. Mol. Biol. 179:125-142, 1984). Genetically encoded hydrophobic amino acids include Gly, Ala, Phe, Val, Leu, Ile, Pro, Met, and Trp, and genetically, encoded hydrophilic amino acids include Thr, His, Glu, Gln, Asp, Arg, Ser, and Lys.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents.

An apolar amino acid is a hydrophobic amino acid with a side chain that is uncharged at physiological pH and which has bonds in which a pair of electrons shared in common by two atoms is generally held, equally by each of the two atoms (i.e., the side chain is not polar). Genetically encoded apolar amino acids include Gly, Leu, Val, Ile, Ala, and Met. Apolar amino acids can be further subdivided to include aliphatic amino acids, which is a hydrophobic amino acid having an aliphatic hydrocarbon side chain. Genetically encoded aliphatic amino acids include Ala, Leu, Val, and Ile.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gln.

An acidic amino acid is a hydrophilic amino acid with a side chain pka value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His.

The above classifications are not absolute, and an amino acid may be classified in more than one category. In addition, amino acids can be classified based on known behavior and or characteristic chemical, physical, or biological properties based on specified assays or as compared with previously identified amino acids. Amino acids can also include bifunctional moieties having amino acid-like side chains.

Conservative changes can also include the substitution of a chemically-derivatized moiety for a non-derivatized residue, by for example, reaction of a functional side group of an amino acid. In addition to the substitutions outlined above, synthetic amino acids providing similar side chain functionality can also be introduced into the self-assembling peptide. For example, aromatic amino acids may be replaced with D- or L-naphthylalanine, D- or L-phenylglycine, D- or L-2-thienylalanine, D- or L-1-, 2-, 3-, or 4-pyrenylalanine, D- or L-3-thienylalanine, D- or L-(2-pyridinyl)-alanine, D- or L-(3-pyridinyl)-alanine, D- or L-(2-pyrazinyl)-alanine, D- or L-p-cyano-phenylalanine, D- or L-(4-isopropyl)-phenylglycine, D- or L-(trifluoromethyl)-phenylglycine, D- or L-(trifluoromethyl)-phenylalanine, D- or L-p-fluorophenylalanine, D- or L-p-biphenylalanine, D- or L-p-methoxybiphenylalanine, D- or L-2-indole (alkyl) alanines, and D- or L-alkylalanines wherein the alkyl group is selected from the group consisting of substituted or unsubstituted methyl, ethyl, propyl, hexyl, butyl, pentyl, isopropyl, iso-butyl, and iso-pentyl.

In an embodiment, the phenylalanine residue(s) present in the self-assembling polypeptide may be replaced a phenylalanine analog. Analogs of phenylalanine include, for example, β-methyl-phenylalanine, β-hydroxyphenylalanine, α-methyl-3-methoxy-DL-phenylalanine, α-methyl-D-phenylalanine, α-methyl-L-phenylalanine, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2,4,5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-cyano-D-phenylalanine, 3-cyano-L-phenylalanine, 3-fluoro-D-phenylalanine, 3-fluoro-L-phenylalanine, 3-iodo-D-phenylalanine, 3-iodo-L-phenylalanine, 3-methyl-D-phenylalanine, 3-methyl-L-phenylalanine, 3-nitro-D-phenylalanine, 3-nitro-L-phenylalanine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-D-phenylalanine, 4-iodo-D-phenylalanine, 4-iodo-L-phenylalanine, homophenylalanine, 1,2,3,4-tetrahydroisoquinoline-3-carboxylic acid (Tic), and 3,3-diphenylalanine. Also, phenylalanine residues may be substituted with tyrosine residues and vice versa.

Analogs of lysine comprising a primary amine in their side chain include ornithine, homolysine, 2,3-diaminoproprionic acid (Dap), and 2,4-diaminobutyric acid (Dab).

Other modifications are also included within the definition of variant of the self-assembling polypeptide of the present disclosure. For example, the size of the self-assembling polypeptide can be reduced by deleting one or more amino acids, and/or amino acid mimetics or dipeptide mimics containing non-peptide bonds may be used. Examples of using molecular scaffolds such as benzodiazepine, azepine, substituted gamma lactam rings, keto-methylene pseudopeptides, β-turn dipeptide cores, and β-aminoalcohols for these purposes are known to peptide chemists and are described in for example Peptidomimetic protocols (Methods in molecular medicine Vol. 23) W. M. Kazmierski (ed.), Humana Press and Advances in Amino Acid Mimetics and Peptidomimetics, Vols. 1 & 2, A. Abell (Ed).

In an embodiment, the self-assembling polypeptide comprises the sequence of SEQ ID NO: 1. In a further embodiment, the self-assembling polypeptide consists of the sequence of SEQ ID NO: 1.

The term “heterologous antigen” as used herein means that the antigen does not comprise domains from the CsgA protein, e.g., the R1, R2, and/or R3 domains of CsgA.

An “immunogen” is meant a molecule that can stimulate the immune system of a host to make a cellular antigen-specific immune response and/or a humoral antibody response when the antigen is presented/administered. It refers to any natural or synthetic compound or chemical entity (lipids, phospholipids, glycolipids, saccharides, nucleic acids, etc.) capable of stimulating an immune response in a host. In an embodiment, the immunogen is a polypeptide (e.g., a protein or peptide derived from a pathogen or a tumor cell). A polypeptide antigen may contain one or more epitope(s). Normally, an epitope will include between about 3-15, generally about 5-15, amino acids. Epitopes of a given protein can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N. J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Mol. Immunol. 23:709-715. Similarly, conformational epitopes are readily identified by determining spatial conformation of amino acids such as by, e.g., x-ray crystallography and 2-dimensional nuclear magnetic resonance (NMR). See, e.g., Epitope Mapping Protocols, supra. “Immunogen” also refers to any natural or synthetic compound or chemical entity (lipids, phospholipids, glycolipids, saccharides, nucleic acids, etc.) capable of stimulating an immune response in a host. Antibodies such as anti-idiotype antibodies, or fragments thereof, and synthetic peptide mimotopes, which can mimic an antigen or antigenic determinant, are also captured under the definition of immunogen as used herein.

The immunogen may be derived from a microorganism or pathogen affecting non-human animals such as pets (cats, dogs) or farm animals (pig, cow, horse, poultry, etc.), or wild animals (such as wild boars, etc.) or humans. In an embodiment, the immunogen is derived from a human pathogen (e.g., a bacteria or a virus affecting humans), or is from human origin (such as a human polypeptide or a fragment thereof).

Further, for purposes of the present disclosure, immunogens (e.g., polypeptides or other biomolecules) can be derived from any of several known pathogens, such as viruses, bacteria, parasites and fungi, as well as any of the various tumor antigens. The immunogen may also be an immunogen involved in diseases or conditions for which vaccination may be useful, e.g., certain allergies and/or immune/inflammation disorders.

The immunogenic construct or composition of the present disclosure contains an immunogen capable of eliciting an immune response against a pathogen, such as an animal or human pathogen, which immunogen may be derived from human immunodeficiency virus (HIV), such as Tat, Nef, Gag, Pol, gp120 or gp160, human herpes viruses, such as gD or derivatives thereof or immediate early protein such as ICP27 from HSV1 or HSV2, cytomegalovirus (such as gB or derivatives thereof), rotavirus, Epstein Barr virus (such as gp350 or derivatives thereof), varicella zoster Virus (such as gpI, II, and IE63), or from a hepatitis virus such as hepatitis B virus (for example hepatitis B surface antigen or a derivative thereof), hepatitis A virus, hepatitis C virus and hepatitis E virus, or from other viral pathogens, such as paramyxoviruses: respiratory syncytial virus (such as F and G proteins or derivatives thereof), parainfluenza virus, measles virus, mumps virus, human papilloma viruses (for example HPV6, 11, 16, 18, etc.), flaviviruses (e.g. yellow fever virus, dengue virus, tick-borne encephalitis virus, Japanese encephalitis virus), influenza virus (e.g., HA, NP, NA, or M proteins, or fragments thereof, or combinations thereof), or coronaviruses (e.g., a SARS-COV-2 antigen, such as the spike [S] glycoprotein or fragments thereof).

Immunogens can also be derived from bacterial pathogens such as Neisseria spp, including N. gonorrhea and N. meningitidis (for example capsular polysaccharides and conjugates thereof, transferrin-binding proteins, lactoferrin binding proteins, PilC, adhesins); Streptococcus pyogenes (for example M proteins or fragments thereof, C5A protease, lipoteichoic acids), S. agalactiae, S. mutans: Haemophilus ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamentous hemagglutinin, adenylate cyclase, fimbriae), B. parapertussis and B. bronchiseptica; Mycobacterium spp., including M. tuberculosis (for example ESAT6, Antigen 85A, -B or -C, Th Ra12, Tb H9, Tb Ra35, Tb38-1, Erd 14, DPV, MTI, MSL, mTTC2, and hTCC1), M. bovis, M. leprae, M. avium, M. paratuberculosis, M. smegmatis; Legionella spp, including L. pneumophila; Escherichia spp., including enterotoxigenic E. coli (for example colonization factors, heat-labile toxin or derivatives thereof, heat-stable toxin, or derivatives thereof), enterohemorrhagic E. coli, enteropathogenic E. coli (for example Shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins, and invasins) and C. coli; Salmonella spp, including S. typhi, S. paratyphi, S. choleraesuis, S. enteritidis; Listeria spp., including L. monocytogenes; Helicobacter spp., including H. pylori (for example urease, catalase, vacuolating toxin); Pseudomonas spp., including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example Clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC. DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the human granulocytic ehrlichiosis; Rickettsia spp., including R. rickettsii; Chlamydia spp. including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leishmania spp., including L. major, Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans, Streptococcus spp., including S. pneumoniae (for example capsular polysaccharides and conjugates thereof, PsaA, PspA, streptolysin, choline-binding proteins) and the protein antigen pneumolysin (Mitchell et al., Biochem Biophys Acta, 1989, 67:1007; Rubins et al., Microb. Pathog., 25:337-342), and mutant detoxified derivatives thereof (WO 90/06951; WO 99/03884), antigens derived from Haemophilus spp., including H. influenzae type B (for example PRP and conjugates thereof), non-typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

Immunogens can also be derived from other microorganisms such as fungi and parasites (protozoa, helminths), including fungi of the genus Candida (Candida albicans, Candida auris), Blastomyces, Cryptococcus (e.g., Cryptococcus gattii, Cryptococcus neoformans), Histoplasma, Coccidioides, Paracoccidioides, Aspergillus (e.g., Aspergillus fumigatus, Aspergillus nidulans, Aspergillus versicolor), and Pneumocystis (e.g., Pneumocystis jirovecii), Taloromyces, parasites of the genus Plasmodium (e.g., Plasmodium vivax, Plasmodium falciparum, Plasmodium malariae, Plasmodium ovale, and Plasmodium knowlesi), Trypanosoma (e.g., Trypanosoma brucei, Trypanosoma cruzi), and Entamoeba (e.g., Entamoeba histolytica), Ascaris lumbricoides, and hookworm.

The conjugate of the present disclosure may also comprise a tumor antigen and be useful for the prevention or immunotherapeutic treatment of cancers. For example, the conjugate may include tumor rejection antigens such as those for prostate, breast, colorectal, lung, pancreatic, renal, or melanoma cancers. Exemplary tumor antigens include MAGE 1, 3, and 4 or other MAGE antigens, PRAME, BAGE, LAGE (also known as NY-Eos-1) SAGE, and HAGE or GAGE. Such antigens are expressed in a wide range of tumor types such as melanoma, lung carcinoma, sarcoma, and bladder carcinoma. Other tumor-specific antigens that may be included in the immunogenic construct or composition of the present disclosure include, but are not restricted to tumor-specific gangliosides such as GM2, GM3, or conjugates thereof to carrier proteins; or said antigen may be a self-peptide hormone such as whole length gonadotrophin hormone releasing hormone, a short 10 amino acid long peptide, useful in the treatment of many cancers. Prostate antigens can also be included, such as prostate specific antigen (PSA), PAP, STEAP, PSCA, PCA3, PSMA, or Prostase. Other tumor-associated antigens (TAA) useful in the context of the present disclosure include: carcinoembryonic antigen (CEA), KSA (also known as EpCAM), gp100, Plu-1, HASH-1, HasH-2, Cripto, and Criptin. Additionally, antigens particularly relevant for vaccines in the therapy of cancer also comprise tyrosinase and survivin. Other antigens include mucin-derived peptides such as Muc1, for example Muc1-derived peptides that comprise at least one repeat unit of the Muc1 peptide, preferably at least two such repeats and which is recognized by the SM3 antibody. Other mucin-derived peptides include peptides from Muc5.

The conjugate of the present disclosure may also comprise antigens associated with tumor-support mechanisms (e.g., angiogenesis, tumor invasion), for example angiopoietin (Ang)-1 and -2, tyrosine kinase with immunoglobulin and epidermal growth factor homology domains (Tie)-2, as well as vascular endothelial growth factor (VEGF).

The conjugate of the present disclosure may also comprise allergen-specific immunogens (for example Der p1, Der p5, grass antigen, Bet v1 (birch), Fel d1 (cats), and allergen non-specific immunogens (for example peptides derived from human IgE, including but not restricted to the Stanworth decapeptide). Other immunogens include for example immunogens derived from Aspergillus fumigatus. Such conjugate may be used for the prophylaxis or therapy of allergy.

In an embodiment, the immunogen is a peptide or a polypeptide, preferably a peptide or a polypeptide of 500 amino acids or less. In an embodiment, the immunogen is a peptide or polypeptide of 400, 350, 300, 250, 200, 150, 100, 90. 80, 70, or 60 amino acids or less. In another embodiment, the immunogen is a peptide of 50, 45, 40, 35, or 30 amino acids or less. In an embodiment, the immunogen is a peptide or polypeptide comprising at least 5, 6, 7, 8, 9, or 10 amino acids. In a further embodiment, the immunogen is a peptide of 10 to 50 amino acids, 15 to 40 amino acids, or 15 to 30 amino acids.

The immunogen may be conjugated to the self-assembling polypeptide directly or indirectly through a linker. For example, the immunogen may be fused directly to the amino-(N) or carboxy (C)-terminal end of the self-assembling polypeptide. In an embodiment, the immunogen is fused to the N-terminal end of the self-assembling polypeptide. In another embodiment, a linker, such as a peptide/polypeptide linker, may be inserted between the immunogen and the self-assembling polypeptide. When the immunogen is a polypeptide and is fused directly to the self-assembling polypeptide or indirectly through a peptide/polypeptide linker, the conjugate may be synthesized as a fusion polypeptide. In an embodiment, the immunogen is indirectly fused to the N-terminal end of the self-assembling polypeptide through a linker.

The molecule (e.g., immunogen) may alternatively be chemically conjugated to the self-assembling polypeptide after expression/synthesis of the self-assembling polypeptide, e.g., before or after self-assembly into nanofilaments.

In another embodiment, the immunogen may be conjugated/attached to the side chain of one the amino acids of the self-assembling polypeptide. Methods for conjugating moieties to side-chains of amino acids are well known in the art. For example, chemical groups that react with primary amines (—NH₂) present in the sidechain of lysine residues such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters may be used to conjugate the immunogen to the self-assembling polypeptide. Most of these groups conjugate to amines by either acylation or alkylation. Cysteine residues present in the self-assembling polypeptide may also be used to attach the immunogen.

The linker of the conjugate may independently be a peptide/polypeptide linker comprising one or more amino acids or another type of chemical linker (e.g., a carbohydrate linker, a lipid linker, a fatty acid linker, a polyether linker, PEG, etc.) having suitable flexibility and stability to allow the conjugate to adopt a proper conformation, e.g., a nanofilament structure. In an embodiment, the linker is a peptide/polypeptide linker. In an embodiment, the peptide/polypeptide linker comprises at least 2 amino acids, and preferably comprises at least 3 or 4 amino acids. The linker may comprise about 100, 90, 80, 70, 60, or 50 amino acids or less, and preferably 20, 15, or 10 amino acids or less. In a further embodiment, the peptide/polypeptide linker comprises about 2 to about 10 amino acids, for example about 4 to about 10 amino acids, or about 6 to about 10 amino acids, for example 7, 8, or 9 amino acids. In an embodiment, the peptide/polypeptide linker is enriched in glycine residues that are known to favor linker flexibility. In an embodiment, the peptide/polypeptide linker comprises one or more serine (Ser or S) and/or threonine (Thr or T) residues, preferably serine residues, which are known to favor linker solubility. In another embodiment, the peptide/polypeptide linker comprises the motif (GGGS) n, wherein n is an integer from 1 to 4. In another embodiment, the peptide/polypeptide linker comprises the sequence GGGSGGGS (SEQ ID NO:25).

In embodiments, the above-mentioned self-assembling polypeptide may comprise, further to the sequence defined above, one or more amino acids (naturally occurring or synthetic) covalently linked to the N- and/or C-terminal end(s) of said polypeptide. In an embodiment, the above-mentioned self-assembling polypeptide comprises up to 5 additional amino acids at the N- and/or C-terminal end(s) of the sequence defined above. In further embodiments, the above-mentioned self-assembling polypeptide comprises up to 5, 4, 3, 2, or 1 additional amino acid(s) at the N- and/or C-terminal end(s) of the sequence defined above. In an embodiment, the above-mentioned self-assembling polypeptide consists of the sequence defined above.

The self-assembling polypeptide or conjugate described herein may further comprise one or more modifications that confer additional biological properties to the conjugate such as protease resistance, plasma protein binding, increased plasma half-life, intracellular penetration, etc. Such modifications include, for example, covalent attachment of molecules/moiety to the conjugate such as fatty acids (e.g., C6-C18), attachment of proteins such as albumin (see, e.g., U.S. Pat. No. 7,268,113); sugars/polysaccharides (glycosylation), biotinylation, or PEGylation (see, e.g., U.S. Pat. Nos. 7,256,258 and 6,528,485). The conjugate may also be conjugated to a molecule that further increases its immunogenicity, including carrier proteins such as keyhole limpet hemocyanin (KLH), bovine serum albumin (BSA), human serum albumin (HSA) and ovalbumin (OVA), and/or polysaccharides. In an embodiment, the conjugate is further conjugated to a carrier protein. The above description of modification of the conjugate does not limit the scope of the approaches nor the possible modifications that can be engineered.

The self-assembling polypeptide or conjugate described herein may be in the form of a salt, e.g., a pharmaceutically acceptable salt. As used herein, the term “pharmaceutically acceptable salt” refers to salts of self-assembling polypeptide or conjugate that retain the biological activity of the parent self-assembling polypeptide or conjugate, and which are not biologically or otherwise undesirable. Such salts can be prepared in situ during the final isolation and purification of the self-assembling polypeptide or conjugate, or may be prepared separately by reacting a free base function with a suitable acid. The self-assembling polypeptides or conjugates disclosed herein are capable of forming acid and/or base salts by virtue of the presence of amino and/or carboxyl groups or groups similar thereto. Pharmaceutically acceptable acid addition salts may be prepared from inorganic and organic acids. Representative acid addition salts include, but are not limited to acetate, adipate, alginate, citrate, aspartate, benzoate, benzenesulfonate, bisulfate, butyrate, camphorate, camphor sulfonate, decanoate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, octanoate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Examples of acids which can be employed to form pharmaceutically acceptable acid addition salts include, for example, an inorganic acid, e.g., hydrochloric acid, hydrobromic acid, sulfuric acid, and phosphoric acid, and an organic acid, e.g., oxalic acid, maleic acid, succinic acid, and citric acid. Basic addition salts also can be prepared by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like. Salts derived from organic bases include, but are not limited to, salts of primary, secondary, and tertiary amines.

The self-assembling polypeptide or conjugate of the disclosure may be produced by expression in a host cell comprising a nucleic acid encoding the self-assembling polypeptide or conjugate (recombinant expression) or by chemical synthesis (e.g., solid-phase peptide synthesis). Peptides and polypeptides can be readily synthesized by manual and automated solid phase procedures well known in the art. Suitable syntheses can be performed for example by utilizing “t-Boc” or “Fmoc” procedures. Techniques and procedures for solid phase synthesis are described in, for example, Solid Phase Peptide Synthesis: A Practical Approach, by E. Atherton and R. C. Sheppard, published by IRL, Oxford University Press, 1989. Alternatively, the polypeptide or conjugate may be prepared by way of segment condensation, as described, for example, in Liu et al., Tetrahedron Lett. 37:933-936, 1996; Baca et al., J. Am. Chem. Soc. 117:1881-1887, 1995; Tarn et al., Int. J. Peptide Prot. Res. 45:209-216, 1995; Schnolzer and Kent, Science 256:221-225, 1992; Liu and Tarn, J. Am. Chem. Soc. 116:4149-4153, 1994; Liu and Tarn, Proc. Natl. Acad. Sci. USA 91:6584-6588, 1994; and Yamashiro and Li, Int. J. Peptide Prot. Res. 31:322-334, 1988). Other methods useful for synthesizing the polypeptides or conjugates are described in Nakagawa et al., J. Am. Chem. Soc. 107:7087-7092, 1985.

Self-assembling polypeptides or conjugates comprising only naturally occurring amino acids encoded by the genetic code may also be prepared using recombinant DNA technology using standard methods. Polypeptides produced by recombinant technology may be modified, e.g., by N-terminal acylation (e.g., acetylation), and/or C-terminal amidation, using methods well known in the art. Therefore, in embodiments, in cases where a self-assembling polypeptides or conjugates described herein contains naturally occurring amino acids encoded by the genetic code, the polypeptide or conjugate may be produced using recombinant methods, and may in embodiments be subjected to for example the just-noted modifications (e.g., acylation, amidation). Accordingly, in another aspect, the disclosure further provides a nucleic acid, such as a DNA or mRNA molecule, encoding the above-mentioned self-assembling polypeptide or conjugate. The disclosure also provides a vector comprising the above-mentioned nucleic acid.

In embodiment, the nucleic acid (DNA, mRNA) encoding the self-assembling polypeptide or conjugate of the disclosure is comprised within a vesicle or nanoparticle such as a lipid vesicle (e.g., liposome) or lipid nanoparticle (LNP), or any other suitable vehicle. Thus, in another aspect, the present disclosure provides a vesicle or nanoparticle, such as a lipid vesicle or nanoparticle, comprising a nucleic acid, such as an mRNA, encoding the self-assembling polypeptide or conjugate described herein.

The term liposome as used herein in accordance with its usual meaning, referring to microscopic lipid vesicles composed of a bilayer of phospholipids or any similar amphipathic lipids (e.g., sphingolipids) encapsulating an internal aqueous medium.

The term “lipid nanoparticle” refers to liposome-like structure that may include one or more lipid bilayer rings surrounding an internal aqueous medium similar to liposomes, or micellar-like structures that encapsulates molecules (e.g., nucleic acids such as mRNA molecules) in a non-aqueous core. Lipid nanoparticles typically contain cationic lipids, such as ionizable cationic lipids. Examples of cationic lipids that may be used for LNPs include DOTMA, DOSPA, DOTAP, ePC, DLin-MC3-DMA, C12-200, ALC-0315, cKK-E12, Lipid H (SM-102), OF-Deg-Lin, A2-Iso5-2DC18, 3060/10, BAME-O16B, TT3, 9A1P9, FTT5, COATSOMER SS-E, COATSOMER SS-EC, COATSOME® SS-OC, and COATSOME® SS-OP (see, e.g., Hou et al., Nat. Rev. Mater., 6:1078-1094, 2021; Tenchov et al., ACS Nano, 15:16982-17015, 2021).

Liposomes and lipid nanoparticles typically include other lipid components such as lipids, lipid-like materials, and polymers that can improve liposome or nanoparticle properties, such as stability, delivery efficacy, tolerability and biodistribution. These include phospholipids (e.g., phosphatidylcholines, phosphatidylethanolamines, phosphatidylserines, and phosphatidylglycerol) such as 1,2-distearoyl-sn-glycero-3-phosphocholine (DSPC) and DOPE, sterols (such as cholesterol and cholesterol derivatives), PEGylated lipids (PEG-lipids) such as 1,2-dimyristoyl-rac-glycero-3-methoxypolyethylene glycol-2000 (PEG₂₀₀₀-DMG), and 1,2-distearoyl-rac-glycero-3-methoxypolyethylene glycol-2000 (PEG₂₀₀₀-DSG).

The nucleic acid (e.g., mRNA) encoding the self-assembling polypeptide or conjugate may be modified, for example to increase stability and/or reduce immunogenicity. For example, the 5′ end may be capped to stabilize the molecule and decrease its immunogenicity (for example, as described in U.S. Pat. Nos. 10,519,189 and 10,494,399). One or more nucleosides of the mRNA may be modified or substituted with 1-methyl pseudo-uridine, to either increase stability of the molecule or reduce recognition of the nucleic acid by the innate immune system. A form of modified nucleosides is described in U.S. Pat. No. 9,371,511. Other types of modifications that may be made to the mRNA include incorporation of anti-reverse cap analog (ARCA), 5′-methyl-cytidine triphosphate (m5CTP), N6-methyl-adenosine-5′-triphosphate (m6ATP), 2-thio-uridine triphosphate (s2UTP), pseudouridine triphosphate, N1-methylpseudouridine triphosphate or 5-methoxyuridine triphosphate (5moUTP). The mRNA may also include additional modifications to the 5′- and/or 3′-untranslated regions (UTRs) and polyadenylation (polyA) tail (see, for example, Kim et al., Mol. Cell. Toxicol. 18:1-8, 2022). All these modifications and other modifications to the nucleic acid (e.g., mRNA) encoding the self-assembling polypeptide or conjugate are encompassed by the present disclosure.

In yet another aspect, the present disclosure provides a cell (e.g., a host cell) comprising the above-mentioned nucleic acid and/or vector. The disclosure further provides a recombinant expression system, vectors, and host cells, such as those described above, for the expression/production of a self-assembling domain or construct of the disclosure, using for example culture media, production, isolation, and purification methods well known in the art.

The self-assembling polypeptide or conjugate of the disclosure can be purified by many techniques of peptide/polypeptide purification well known in the art, such as reverse phase chromatography, high performance liquid chromatography (HPLC), ion exchange chromatography, size exclusion chromatography, affinity chromatography, gel electrophoresis, and the like. The actual conditions used to purify a particular peptide or polypeptide will depend, in part, on synthesis strategy and on factors such as net charge, hydrophobicity, hydrophilicity, and the like, and will be apparent to those of ordinary skill in the art. For affinity chromatography purification, any antibody that specifically binds the peptide/polypeptide may for example be used.

The present disclosure also provides compositions, such as pharmaceutical compositions and vaccines, comprising the self-assembling polypeptide, conjugate, or nucleic acid described herein. In an embodiment, the composition further comprises one or more pharmaceutically acceptable carrier(s), excipient(s), and/or diluent(s). In an embodiment, the composition (e.g., vaccine) further comprises a pharmaceutically acceptable vaccine adjuvant. In another embodiment, the composition (e.g., vaccine) is free of pharmaceutically acceptable vaccine adjuvant, i.e., the conjugate is inherently sufficiently immunogenic to induce a suitable immune response in the absence of an additional vaccine adjuvant in the vaccine.

As used herein, “pharmaceutically acceptable” (or “biologically acceptable”) refers to materials characterized by the absence of (or limited) toxic or adverse biological effects in vivo. It refers to those compounds, compositions, and/or dosage forms which are, within the scope of sound medical judgment, suitable for use in contact with the biological fluids and/or tissues and/or organs of a subject (e.g., human, animal) without excessive toxicity, irritation, allergic response, or other problem or complication, commensurate with a reasonable benefit/risk ratio.

The term “vaccine adjuvant” refers to a substance which, when added to an immunogenic agent such as an antigen (e.g., the conjugate, nucleic acid, or composition defined herein), non-specifically enhances or potentiates an immune response to the agent in the host upon exposure to the mixture. Suitable vaccine adjuvants are well known in the art and include, for example: (1) mineral salts (aluminum salts such as aluminum phosphate, aluminum hydroxide, and calcium phosphate gels), squalene, (2) oil-based adjuvants such as oil emulsions and surfactant based formulations, e.g., incomplete or complete Freud's adjuvant, MF59 (microfluidised detergent stabilised oil-in-water emulsion), QS21 (purified saponin), AS02 ([SBAS2] oil-in-water emulsion+MPL+QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), complete Freud's adjuvant (comprising inactivated and dried mycobacteria) (5) endogenous human immunomodulators, e.g., hGM-CSF or hIL-12 (cytokines that can be administered either as protein or plasmid encoded), Immudaptin (C3d tandem array), and/or (6) inert vehicles, such as gold particles.

An “excipient” as used herein has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents, and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present disclosure is not limited in these respects. In certain embodiments, the composition of the present disclosure include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents, and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipients include water, saline, phosphate buffered saline, dextrose, glycerol, ethanol, and the like, as well as combinations thereof. In many cases, it will be preferable to include isotonic agents, for example, sugars, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness.

The composition (e.g., vaccine) of the present disclosure may be formulated for administration via any conventional route, such as intravenous, oral, transdermal, intraperitoneal, subcutaneous, mucosal, intramuscular, intranasal, intrapulmonary, intrarectal, intraocular, parenteral, or topical administration. The preparation of such formulations is well known in the art (see, e.g., Remington: The Science and Practice of Pharmacy, Lippincott Williams & Wilkins; 21^{st} edition, 2005). For administration to non-human mammals, formulations for administration in ovo or in the mammary gland administration may be considered. In an embodiment, the composition of the present disclosure is formulated for administration by injection, for example intravenous, subcutaneous, intradermal, or intramuscular administration. In another embodiment, the composition of the present disclosure is formulated for intranasal or pulmonary (e.g., aerosol) administration.

The conjugate, nucleic acid, composition, or vaccine defined herein may be used in biomedical applications.

In another aspect, the present disclosure also provides a method for delivering an immunogen (such as one or more of the immunogens defined above) in a subject comprising administering to the subject an effective amount of the conjugate, nucleic acid, composition, or vaccine defined herein.

In another aspect, the present disclosure also provides a method for inducing an immune response against an immunogen (e.g., one or more of the immunogens defined above) in a subject comprising administering to the subject an effective amount of the conjugate, nucleic acid, composition, or vaccine defined herein. The present disclosure also provides the use of the conjugate, nucleic acid, composition, or vaccine defined herein for inducing an immune response against an immunogen (e.g., one or more of the immunogens defined above) in a subject. The present disclosure also provides the use of the conjugate, nucleic acid, composition, or vaccine defined herein defined herein for the manufacture of a medicament for inducing an immune response against an immunogen (e.g., one or more of the immunogens defined above) in a subject. The present disclosure also provides the conjugate, nucleic acid, composition, or vaccine defined herein for inducing an immune response against an immunogen (e.g., one or more of the immunogens defined above) in a subject. The present disclosure also provides the conjugate, nucleic acid, composition, or vaccine defined herein for use as a medicament.

In another aspect, the present disclosure also provides a method for preventing and/or treating a microbial infection or cancer in a subject comprising administering to the subject an effective amount of the conjugate, nucleic acid, composition, or vaccine defined herein. The present disclosure also provides the use of the conjugate, nucleic acid, composition, or vaccine defined herein for preventing and/or treating a microbial infection or cancer in a subject. The present disclosure also provides the use of the conjugate, nucleic acid, composition, or vaccine defined herein for the manufacture of a medicament for preventing and/or treating a microbial infection or cancer in a subject. The present disclosure also provides the conjugate, nucleic acid, composition, or vaccine defined herein for use in preventing and/or treating a microbial infection or cancer in a subject.

Any suitable amount of the conjugate, nucleic acid, composition, or vaccine defined herein may be administered to a subject. The dosages will depend on many factors including the mode of administration. Typically, the amount of conjugate, nucleic acid, composition, or vaccine defined herein contained within a single dose will be an amount that effectively induces an immune response against an immunogen, and/or prevent, delay, or treat a microbial infection or cancer without inducing significant toxicity. For the prevention, treatment, or reduction in the severity of a given disease or condition, the appropriate dosage of the conjugate, nucleic acid, composition, or vaccine will depend on the type of disease or condition to be treated, the severity and course of the disease or condition, whether the conjugate, nucleic acid, composition, or vaccine is administered for preventive or therapeutic purposes, previous therapy, the patient's clinical history and response to the conjugate, nucleic acid, composition, or vaccine, and the discretion of the attending physician. The conjugate, nucleic acid, composition, or vaccine is suitably administered to the patient at one time or over a series of treatments. Preferably, it is desirable to determine the dose-response curve in vitro, and then in useful animal models prior to testing in humans. The present disclosure provides dosages for the conjugate, nucleic acid, composition, or vaccine. For example, depending on the type and severity of the disease, a dosage of about 1 μg/kg to 1000 mg per kg (mg/kg) of body weight per day may be administered. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs.

The administration/use may be performed prophylactically, i.e., prior to the development of the infection or disease, or therapeutically in a subject suffering from a disease or infected with a pathogen.

### EXAMPLES

The present disclosure is illustrated in further details by the following non-limiting examples.

### Example 1: Materials and Methods

Protein Expression, Purification and Self-Assembly. The pET-29a (+) plasmids containing the 3M2e-CsgA, 3M2e-R4R5, eGFP-CsgA, eGFP-R4R5, and 3M2e sequences were generated from GeneScript services. The N-terminal secretion signal was removed from the CsgA sequence (Genbank accession number: WP_074524256.1). The M2e sequence derived from the A/Puerto Rico/8/1934/H1N1 IAV strain, with the two cysteine residues replaced by serines (SLLTEVETPIRNEWGSRSNGSSD, SEQ ID NO:2) to avoid the formation of disulfide bond without affecting immunogenicity, as previously reported⁹⁹. The tandem repeats of M2e were spaced by a flexible linker (GGGSGGGS, SEQ ID NO:25) and a HisTag (6×His) was inserted at the C-terminus.

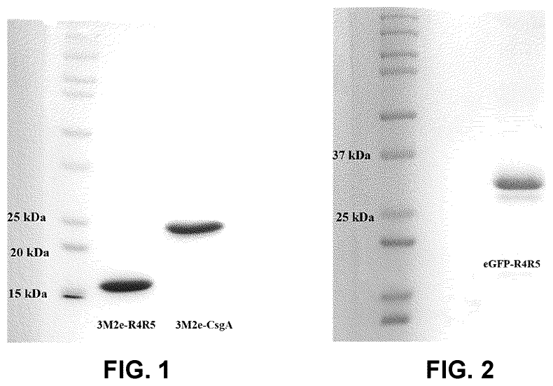

Plasmids were transformed in E. coli NiCo21(DE3) (New England Biolabs) and cells were grown to an optical density (O.D.) at 600 nm of 1.0 before inducing protein expression with 0.5 mM isopropyl-thiogalactopyranoside (IPTG) for 1 h at 37° C. Cells were then harvested. For purification of 3M2e-CsgA and 3M2e-R4R5, cells were lysed with 8 M guanidium hydrochloride (Gdn-HCl) followed by sonication and centrifugation to pellet large debris. Nickel resin Profinity™ immobilized metal-ion affinity chromatography (IMAC; Bio-Rad) was added to the cell lysate and incubated for 1 h while tumbling at RT. The lysate was passed through a column and the resin was washed with 10 column volumes (CVs) of ice-cold PBS and then 10 CVs of ice-cold phosphate buffered saline (PBS) supplemented with 12.5 mM of imidazole. Finally, the proteins were eluted with 250 mM imidazole and desalted in PBS buffer with Sephadex™ G-25 fine beads by centrifugation. The protein solutions were sterilized by filtration with 0.2 μm polyvinylidene fluoride (PVDF) filter. Protein concentrations were determined with bicinchoninic acid (BCA) reagent and purity of proteins were verified by SDS-PAGE (FIG. 1). Before immunization, endotoxins were removed with Pierce endotoxin-removal spin columns. Freshly purified proteins were assembled at RT in PBS buffer without agitation for 24 h at a concentration of 600 μg/mL.

For eGFP-CsgA, eGFP-R4R5 and 3M2e, cells were lysed with ice-cold PBS supplemented with 1% (v/V) Triton™ X-100, 5% (v/v) glycerol, 25 mM of sucrose, 1 mM of ethylenediaminetetraacetic acid (EDTA), complete-mini protease inhibitor, Pierce's nuclease, and lysozyme followed by sonication (4×20 seconds) on ice and centrifugation at 4° C. to pellet debris. Nickel resin Profinity™ immobilized metal-ion affinity chromatography (IMAC) was added to the lysate and incubated 1 h while tumbling at 4° C. Purification process was as previously detailed. Protein concentrations were determined with BCA reagent and purity of proteins were verified by SDS-PAGE (FIG. 2). Freshly purified proteins were assembled at RT in PBS buffer without agitation for 24 h at a concentration of 600 μg/mL.

Transmission Electron Microscopy. Protein solutions were sonicated and diluted to 60 μg/mL in H₂O before being applied to a glow-discharged 300 mesh copper carbon-coated grid. Samples were dried and stained with 1.5% (w/v) uranyl formate for 1 min before air-drying. Grids were imaged using a FEI Tecnai™ G2 Spirit Twin microscope at 120 kV and mounted with a Gatan Ultrascan™ 4000 4k×4k CCD camera system.

Circular Dichroism Spectroscopy. Protein solutions were diluted to 200 μg/mL with H₂O and added to a 1 mm pathlength quartz cell for analysis with a Jasco™ J-815 CD spectropolarimeter. Measurement was set every 0.5 nm between 260 nm and 190 nm with a scan rate of 10 nm/min. Background was subtracted with the PBS buffer alone and the spectra were smoothed with the Savitsky-Golay algorithm at 11 points. Raw data were converted to mean residue ellipticity (MRE) using this formula:

$$M R E ( deg {cm}^{2} {dmol}^{- 1} ) = \frac{CD signal ( mdeg ) \times {10}^{5}}{\begin{matrix}pathlenght ( cm ) \times protein concentration ( μM ) \times \\ number of residues\end{matrix}}$$

Fluorescence Spectroscopy. Thioflavin T was added to the protein samples at a final concentration of 40 μM and the fluorescence emission was measured between 450 nm and 550 nm with constant excitation at 440 nm in a QuantaMaster™ 40 spectrofluorometer. 8-anilino-1-naphthalenesulfonic acid (ANS) was added to the protein samples at a final concentration of 450 UM and the fluorescence emission was measured between 385 nm and 550 nm with constant excitation at 370 nm.

Measurement of Solution Turbidity. Turbidity of protein solutions was measured between 600 nm and 400 nm in a 10 mm pathlength quartz cell with a UV-1280 Shimadzu™ spectrophotometer.

Measurement of Viscosity. Analysis of solution viscosity was performed with the SV-10 viscosimeter at RT in 10-mL cells. The 5% (v/v) glycerol solution was prepared in PBS and extensively mixed right before analysis.

Determination of Antigen Accessibility by enzyme-linked immunosorbent assay (ELISA). High binding 96-well microplates (ThermoFisher Scientific) were coated overnight with 1 μM of CsgA-based assemblies or 3M2e in 50 mM sodium carbonate buffer pH 9.6 at 4° C. Following washing with PBS+0.05% Tween™ 20 (PBS-Tween™), wells were blocked for 1 h with 1% w/v of bovine serum albumin (BSA) in PBS-Tween™ at RT. Wells were washed with PBS-Tween™ and incubated 2 h at RT with 2-fold dilutions of anti-influenza M2 14C2 monoclonal antibody starting at a 1:250 dilution in blocking buffer. Plates were washed with PBS-Tween™ and incubated 1 h at RT with horseradish peroxidase (HRP)-conjugated goat anti-mouse IgG at a dilution of 1:20,000 in blocking buffer. Plates were extensively washed with PBS-Tween™ and 3,3′-5,5′-tetramethyl benzidine (TMB) substrate was added for 15 min at RT. The reaction was stopped with 1 N sulfuric acid (H₂SO₄) and the optical density at 450 nm was measured. Data are presented as dilution of primary antibody.

Evaluation of TLR2-TLR1 Activation. HEK-Blue hTLR2-TLR1 cells (InvivoGen) were cultured in DMEM supplemented with 4.5 g/L of glucose, 2 mM of L-glutamine, 10% (v/v) of fetal bovine serum (FBS), 100 U/mL of penicillin-streptomycin and 100 μg/mL of Normocin at 37° C. under 5% CO₂. At roughly 80% confluence, cells were seeded in 96-well plates at a density of 50,000 cells/well in HEK-Blue detection medium and incubated 16 h with protein solutions at the indicated concentrations. The colorimetric reaction was evaluated by measuring the absorbance at 620 nm.

Secretion IL-1β by Macrophages. J774.A1 macrophages were cultured in DMEM supplemented with 10% (v/v) of FBS and 100 U/mL of penicillin-streptomycin at 37° C. under 5% CO₂. Cells were seeded in 24-well plates at a density of 100,000 cells/well and incubated 16 h with CsgA-based assemblies and respective controls. Supernatants were collected and the amount of IL-1β was determined by sandwich ELISA according to the manufacturer's procedures (ThermoFisher Scientific).

Evaluation of Cell Viability. For metabolic activity, J774.A1 cells were seeded in 96-well plates at a density of 30,000 cells/well and incubated 16 h with CsgA-based assemblies. Resazurin (50 μM) was added and after 4 h incubation, the absorbance at 570 nm was measured. Cell viability (in %) was calculated from the ratio of the fluorescence of the treated sample to the PBS control treated cells. For LIVE/DEAD assay, J774.A1 cells were seeded the day prior in 8-chamber cover glass at a density of 100,000 cells/chamber and the following day cells were incubated overnight with CsgA-based assemblies or controls. Cell medium was removed and cells were stained with 4 μM of ethidium homodimer and 2 μM of calcein-AM (ThermoFisher Scientific) in PBS for 30 min at RT before imaging on a Nikon A1R confocal microscope. Dead cells were generated following 10 minutes treatment with 70% methanol.

Evaluation of Cellular Uptake. J774.A1 were seeded in 24-well plates at a density of 200,000 cells/well. After overnight incubation, soluble eGFP and eGFP-R4R5 were respectively added to the cell media and cells were incubated at 37° C. for 3 h before extensive washing with PBS. Cells were analyzed in a BD Accuri™ flow cytometer with excitation at 488 nm and emission at 525 nm following quenching with 50% (v/v) of trypan blue to remove membrane-associated fluorescence. The FlowJo™ program was used to determine eGFP median fluorescence intensity.

For fluorescence confocal microscopy, J774.A1 macrophages and DC2.4 cells were seeded in 8-chamber cover glass at a density of 50,000 cells/chamber. After overnight incubation, cells were respectively incubated with soluble eGFP and eGFP-R4R5 for 3 h before extensive washing with PBS. Cells were fixed with 4% formaldehyde for 10 min and stained for 30 min at RT with 0.5 μg/mL 4′,6-diamidino-2-phenylindole dihydrochloride (DAPI) and 0.165 μM Texas Red-X phalloidin (ThermoFisher Scientific). Cells were washed and imaged in a Nikon A1R confocal microscope with a 60× oil immersion lens (405, 488, and 562 nm laser excitation). Images were analyzed using the ImageJ software and presented as Z-stack.

Activation of Dendritic cells. DC2.4 cells were seeded in 24-well plates at a density of 200,000 cells/well and treated for 24 h with CsgA-based assemblies. Cells were washed with FACS buffer (PBS 2% FBS [v/v], 2 mM EDTA) and incubated 30 minutes in Fc block (2.4G2 hybridoma supernatant). Cells were stained in FACS buffer with anti-mouse MHCII (M5/114.15.2) PE-Cy5 monoclonal antibody and anti-CD80 (16-10A1) PE-Cy7 monoclonal antibody (eBioscience) at 1 μg/mL for 45 minutes and then fixed for 10 min as previously mentioned. Fluorescence was measured using a Beckman Coulter CytoFLEX™ cytometer and data was analyzed using FlowJo™ software.

Mice Immunization (intramuscular). Six-to-eight weeks old female BALB/c mice (Charles River) were placed under isoflurane anesthesia and immunized intramuscularly with 18 μg of 3M2e or equimolar doses of 3M2e-CsgA (50 μg) or 3M2e-R4R5 (30 μg) diluted in 100 μL of endotoxin-free PBS, or 50 μL of PBS and 50 μL of Alum (1:1) for the Alum-supplemented formulations. Weights were monitored every day following immunization. Three immunizations were performed 14 days apart and sera were collected the day prior to the immunization via the saphenous vein. In each group, four out of 12 mice were sacrificed 13 days after the second boost to collect blood by cardiac puncture for antibodies isotyping. The sera of the other 8 mice were collected via the saphenous vein 13 days after the third immunization.

Mice Immunization (intranasal). Groups of 6-week-old female Balb/C mice were immunized intranasally by nasal instillation twice at two weeks interval with 20 μg/mL of R4R5-3M2e or the molar equivalent of 3M2e peptide in a total volume of 50 μL in endotoxin-free PBS (Millipore Sigma). Control mice were inoculated with endotoxin-free PBS alone. Procedures were performed under light isoflurane anaesthesia.

Antibody Titers by ELISA. High-binding ELISA plates were coated overnight with 2 μg/mL of M2e synthetic peptide in 50 mM sodium carbonate buffer pH 9.6 at 4° C. Following washing with PBS-Tween™, wells were blocked for 1 h with blocking buffer at RT. Plates were washed with PBS-Tween™ and incubated 2 h at RT with 2-fold dilutions of mice sera starting at a 1:65 dilution in blocking buffer. Plates were washed with PBS-Tween™ and incubated 1 h at RT with HRP-conjugated goat anti-mouse IgG at a dilution of 1:20,000 in blocking buffer. After washing with PBS-Tween™, TMB substrate was added for 15 minutes at RT. The reaction was stopped with 2 N H₂SO₄ and the O.D. at 450 nm was measured. The O.D. at 450 nm was plotted against dilution of sera by means of a regression curve: (y=[b+cx])/[1+ax]). The antibody titer was established as the highest sera dilution associated with an absorbance value twice that of the blank control (blocking buffer only). For IgG isotypes (IgG1, IgG2a, IgG2b, and IgG3), HRP-conjugated goat anti-mouse antibodies were diluted 1:30,000. Data is represented as O.D. at 450 nm at a given dilution (IgG1=1:16,000; IgG2a=1:1000; IgG2b=1:1000; IgG3=1:65).

ELISPOT Assay. Spleen were collected from immunized mice seven days after the third dose and splenocytes were extracted with a 70 μm cell-strainer and red blood cells were lysed using red blood cells lysis buffer (Sigma). Mouse IFNγ/IL-4 dual color ELISpot kit (ImmunoSpot CTL) were used to measure the production of both cytokines following ex vivo stimulation. ELISpot 96-well plates (Millipore) were pre-treated with 70% ethanol and coated overnight with capture antibodies. Wells were washed with PBS and blocked with splenocytes media (RPMI-1640 supplemented with 10% FBS, 2 mM L-glutamine, 1 mM HEPES, 4.5 g/L of glucose, 1.5 g/L of sodium bicarbonate, 50 M of 2-mercaptoethanol and 100 U/mL of penicillin-streptomycin) for 2 h at 37° C. Splenocytes were seeded at a density of 500,000 cells/well in 96-well ELISpot plate (Millipore) and stimulated with 10 μg/ml of M2e synthetic peptide for 36 h at 37° C. under 5% CO₂. Spots were detected by alkaline phosphatase (AP)-conjugated (IL-4) or HRP-conjugated (IFNγ) detection antibodies and the number of spots was calculated with an ELISpot plate reader (ImmunoSpot CTL). Control splenocytes were stimulated with 10 μg/mL of the E2EP3 peptide, an epitope derived from the Chikungunya virus, or with a combination of 250 ng/ml of phorbol 12-myristate 13-acetate (PMA) and 500 ng/ml of ionomycin.

Secretion of IFNγ and IL-4 from isolated splenocytes. Isolated splenocytes, as above-described, were seeded in 96-well tissue culture treated plate and stimulated with 10 μg/mL of M2e synthetic peptide. After 72 h stimulation, the supernatants were collected to quantify cytokine production using sandwich ELISA for IFNγ and IL-4 (ThermoFisher Scientific).

Recovery of spleen, lungs, BALF and NALT. Two weeks after the second intranasal immunization, mice were euthanized by cervical dislocation under isoflurane anesthesia and whole blood, spleens, lungs, broncho-alveolar lavage fluid (BALF) and nasal associated lymphoid tissue (NALT) were recovered. For BALF recovery, trachea was exposed with surgical tools and a small incision was made. A homemade cannula attached to a 1-ml syringe was introduced in the trachea and 1 mL of ice-cold endotoxin free PBS was injected in the lungs. An average of 700 μL was recovered per mice and was centrifuged at 800 g for 5 min to pellet cells. BALF and sera were stored at 80° C. until assayed. For NALT recovery, palate from mice were removed surgically and serially washed with DMEM (Wisent) supplemented with 10% FBS and 100 U/mL antibiotics. Afterward, NALT were incubated in 250 μL complete media for 48 hours after which media was collected. Cells were removed by centrifugation (12000 g, 5 minutes) and supernatant was stored at −80° C. until assayed.

Influenza A Experimental Challenge. Fourteen days after the third immunization, mice were anesthetized with isoflurane and instilled with 5×LD₅₀ of influenza virus A/Puerto Rico/8/1934/H1N1. Weight and clinical scores were monitored twice every day (Table 1). Mice that lost more than 20% of their initial weight, or that showed clinical signs of severe symptoms, were sacrificed by isoflurane inhalation and cervical dislocation.

TABLE 1
Scale for clinical symptoms of influenza infection

| | Temperature | | | | |
|---|---|---|---|---|---|
| Intensity | (° C.) | Fur | Posture | Eyes | Ears |
| 0 | >36 | Smooth and | Normal | Open | Normal |
| (absent) | | even | | | |
| 1 | 35-36 | Fur loss | Slightly | Half- | Bent |
| (light) | | | hunched | closed | |
| 2 | 32-35 | | back | | |
| (moderate) | | | | | |
| 3 | <32 | Ruffled fur | Hunched | Closed | Laid down |
| (severe) | | | back | | |

| | Response to | | | Dehydration |
|---|---|---|---|---|
| Intensity | stimuli | Activity | Feces | (Pinch on skin) |
| 0 | Normal | Normal | Normal | Normal |
| (absent) | | | | |
| 1 | Calm, curious | Reduced | Soft | Skin rapidly |
| (light) | | | | recovers |
| 2 | Delay in | Immobile, | Sticky | Skin slowly |
| (moderate) | response | reactive | | recovers |
| 3 | Inactive | Lethargic | Liquid | No recovery |
| (severe) | | | | |

Evaluation of Pro-Inflammatory Response. Six-to-eight weeks old female BALB/c mice (Charles River) were inoculated intraperitoneally with 20 μM of CsgA-based filaments in 50 μL of endotoxin-free PBS or same concentration of controls. Before immunization, endotoxins were removed with Pierce endotoxin-removal spin columns. FljB-3M2e chimeric protein was obtained as previously described¹⁰⁰. Weight and rectal temperature were monitored at 2, 6, and 24 h post-inoculation and mice were sacrificed, after cardiac puncture under isoflurane anesthesia, by cervical dislocation. Levels of IL-6 and TNFα in the sera was measured using sandwich ELISA kits (ThermoFisher Scientific).

### Example 2: Design and Characterization of CsgA-Based Filaments

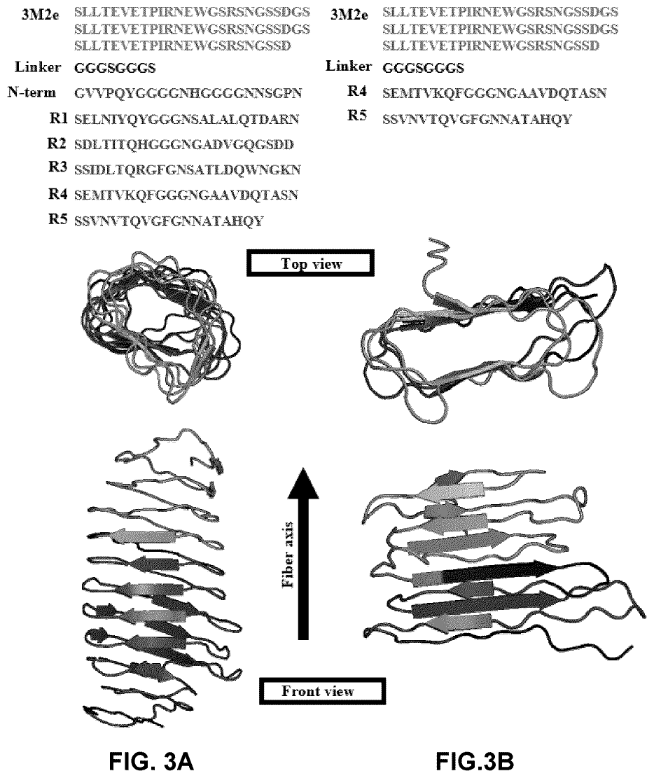

The ectodomain of the IAV M2 protein, M2e, is a potential linear epitope-containing peptide candidate for an universal flu vaccine, owing to its highly conserved sequence amongst various IAV subtypes⁵⁵. Although it is expressed at the surface of the virions, M2e is not neutralizing per se, but it is targeted by antibodies at the surface of IAV-infected cells and promotes their elimination via antibody-dependent cellular phagocytosis (ADCP) by alveolar macrophages (AM) 56 and antibody-dependent cellular cytotoxicity (ADCC) by natural killer (NK) cells^{57,58}. However, M2e peptide is poorly immunogenic compared to hemagglutinin (HA) and delivery strategies are needed to enhance its immunogenicity. Three repetitions of H1N1 IAV M2e (SLLTEVETPIRNEWGSRSNGSSD, SEQ ID NO:2) were fused to the N-terminal domain of full-length CsgA, and flexible linkers (GGGSGGGS) were added between the 3M2e and the self-assembling units (FIG. 3A). A truncated version of CsgA containing only the fourth and fifth repeating units (R4R5) was also evaluated (FIG. 3B). Both chimeric proteins displaying a C-terminal 6×His-Tag were expressed in E. coli and purified under denaturing conditions by immobilized metal affinity chromatography (IMAC) yielding two proteins of 23 kDa and 14 kDa for 3M2e-CsgA and 3M2e-R4R5, respectively (FIG. 1).

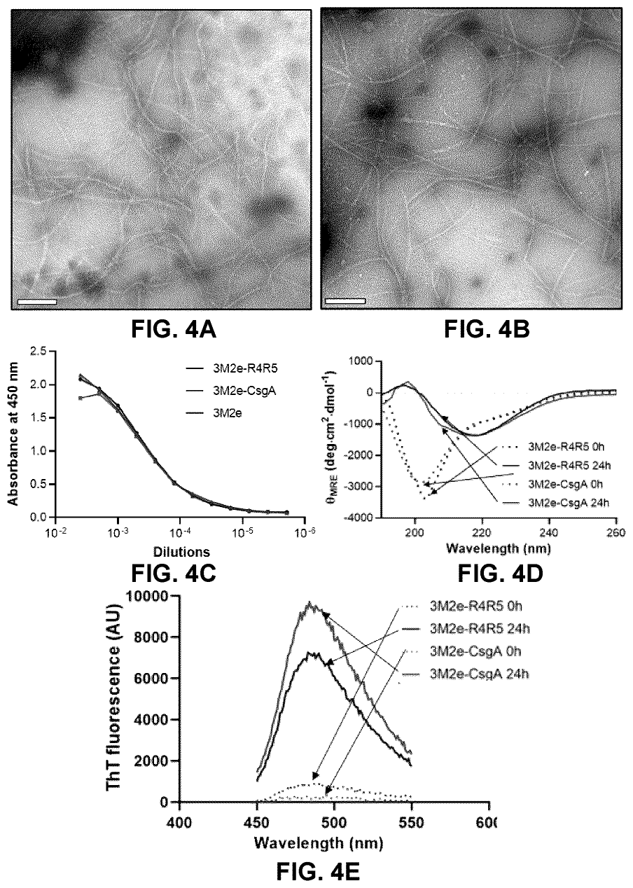

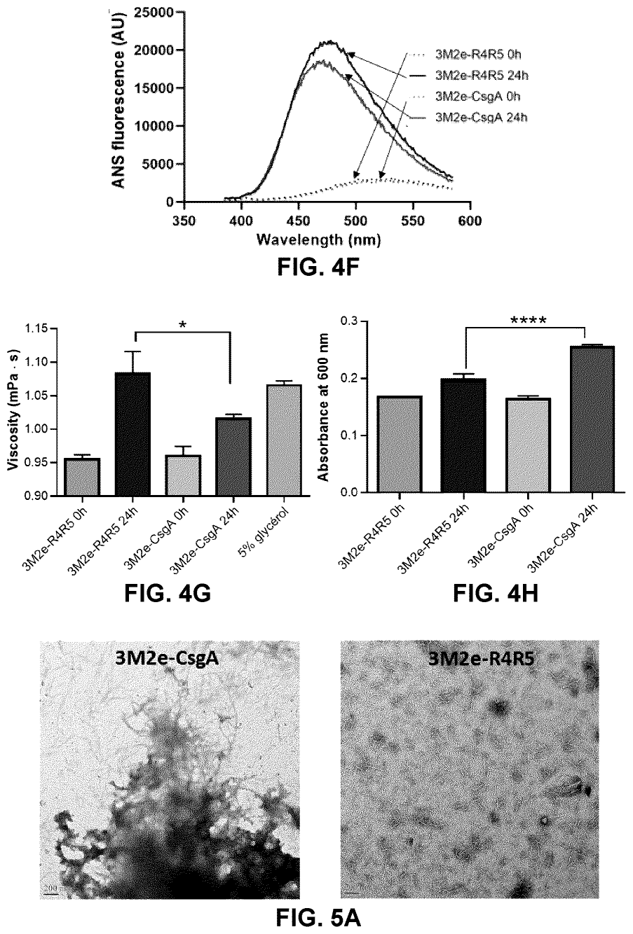

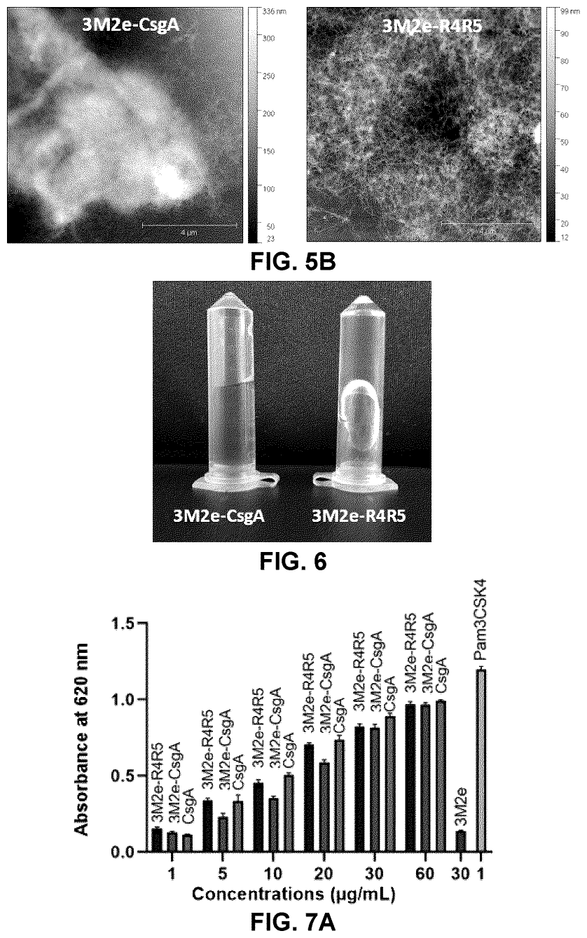

After the removal of denaturing agents, freshly purified 3M2e-CsgA and 3M2e-R4R5 were left at RT under fully quiescent conditions for 24 h at a concentration of 600 μg/mL in PBS to initiate self-assembly. Negative stain transmission electron microscopy (TEM) showed that both proteins assembled into linear and thin filaments of up to a few micrometers in length with a diameter of 5 to 10 nm (FIGS. 4A and 4B), with morphology similar to what has been previously reported for CsgA assemblies⁴¹. Nonetheless, it is worth noting that large clumps of nanofilaments were observed by atomic force microscopy (AFM) and TEM for full length CsgA preparations. Such clumps were not observed in 3Me2-R4R5 solutions in which nanofilaments remained in suspension (FIGS. 5A and 5B). An anti-M2e enzyme-linked immunosorbent assay (ELISA) confirmed that the antigen was accessible to antibodies and maintained its antigenicity following assembly into filaments (FIG. 4C). Circular dichroism spectroscopy revealed that both 3M2e-CsgA and 3M2e-R4R5 were mostly unstructured immediately after their purification and shifted to a β-sheet-rich secondary structure after 24 h incubation, as exemplified by a shift from a spectrum characterized with a single minimum at around 200 nm to a spectrum with minimum at 220 nm and a maximum at around 198 nm (FIG. 4D). Such conformational conversion is a prototypical characteristic of the self-assembly of CsgA-based proteins^{41,59,60}. Analysis of thioflavin T (ThT) fluorescence, a small dye that binds selectively to cross-β-sheet motif⁶¹, further confirmed self-assembly (FIG. 4E). Moreover, the dye anilinonaphthalene sulfonic acid (ANS), whose fluorescence quantum yield increases upon binding to protein hydrophobic domains, revealed the formation of accessible hydrophobic pockets upon formation of fibrillar assemblies (FIG. 4F). Protein self-recognition and aggregation are known to increase the solution viscosity, a property that can be beneficial for vaccine formulations by increasing retention time at the site of injection and on mucosal surface⁶²⁻⁶⁴. Accordingly, the viscosity of 3M2e-CsgA and 3M2e-R4R5 solutions was analyzed immediately after purification, under their monomeric form, and after 24 h incubation. Both protein solutions showed an increase in viscosity associated with the self-assembly process, with the 3M2e-R4R5 solution being significantly more viscous compared to 3M2e-CsgA (FIGS. 4G and 6). In fact, the solution of 3M2e-R4R5 filaments at a concentration of 600 μg/mL had comparable viscosity to a 5% glycerol solution. Additionally, the colloidal properties of protein assemblies are known to increase visible light scattering, and turbidity is often used to follow protein aggregation in aqueous solutions. At a concentration of 600 μg/mL, an increase of absorbance at 600 nm was observed after self-assembly, with the 3M2e-CsgA solution being significantly more turbid (FIG. 2H). Overall, these results indicate that the assembly of CsgA and its truncated R4R5 fragment tolerates the addition of antigen at their N-terminus and that the M2e epitope is accessible at the filament surface. Moreover, deleting the R1 to R3 segments of CsgA, i.e., R4R5, led to a suspension of well-defined nanofilaments that remained in solution; the increased viscosity could potentially led to an improved efficacy of the vaccine formulation.

### Example 3: CsgA Nanofilaments Activate TLR2-TLR1 and Promote IL-1β Secretion

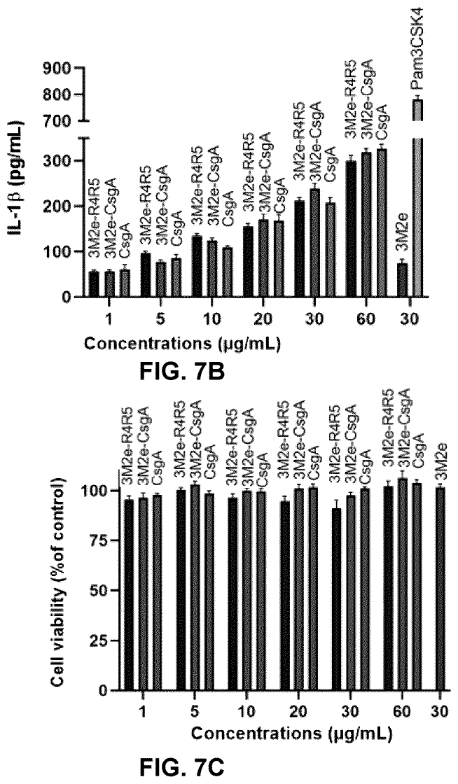

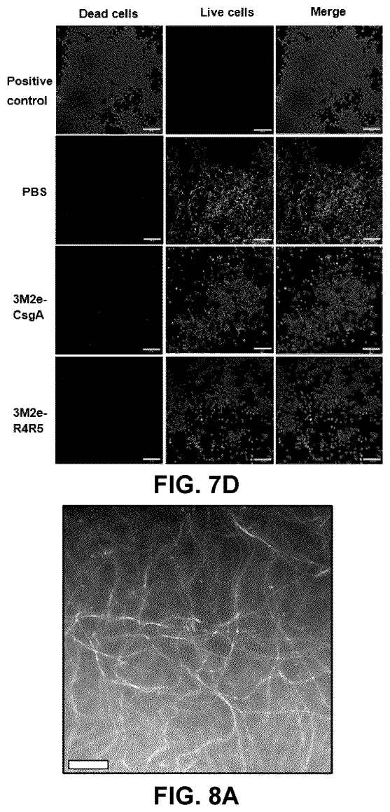

Toll-like receptor (TLR) signaling is involved in the activation and maturation of immune cells and in the induction of a robust antigen-specific immune response⁶⁵. TLR2 is a cell surface receptor that forms heterodimers with TLR1 or TLR6, and is widely expressed on APCs and endothelial cells^{66 67}. TLR2 recognizes a large diversity of ligands, including lipoproteins, peptidoglycans, porins, and the cross-β quaternary motif of protein assemblies^{50,68,69}. Supplementing vaccine formulations with TLR2 agonist, such as Pam2CSK470 or Pam3CSK471, has been shown to promote the induction of a pro-inflammatory environment that favors the recruitment of immune cells and the maturation of APCs and their migration to the lymph nodes, ultimately inducing a robust cellular and humoral immune response^{68,69,72-74}. CsgA fibrils are known to engage the heterodimer TLR2-TLR1 in bone-marrow derived macrophages^{48,50}. To validate that the addition of the 3M2e antigenic sequence did not preclude activation of TLR2-TLR1, the HEK-Blue TLR2-TLR1 reporter cell line, which express a NF-κB/AP-1-inducible secreted embryonic alkaline phosphatase (SEAP) reporter, were used to monitor TLR2 signaling. Assemblies of 3M2e-CsgA and 3M2e-R4R5 showed comparable concentration-dependent activation of TLR2-TLR1-mediated NF-κB signaling, which was equivalent to the signal obtained with unmodified CsgA filaments (FIG. 7A). This result indicates that the addition of an antigen at the surface of the CsgA filaments does not affect its TLR2-agonist properties. Following binding and activation of the TLR2-TLR1, the receptor and ligand are conjointly endocytosed, and a fraction of the fibrils can leak out of the endosomes into the cytosol and activates the NLRP3 inflammasome, leading to production of pro-IL-1β and subsequent cleavage into IL-β⁴⁷. To assess IL-1β secretion associated with potential inflammasome activation, macrophages J774.A1 were exposed to increasing concentration of fibrils and the level of IL-1β in the cell media obtained 16 h after treatment was measured by ELISA. The three CsgA nanoassemblies (CsgA, 3M2e-CsgA and 3M2e-R4R5) induced an equivalent concentration-dependent production of IL-1ß by macrophages, suggesting that the inflammasome activating properties were retained upon N-terminal conjugation of the antigenic peptides⁷⁵ (FIG. 7B). It is known that IL-1β secretion can be associated with pyroptosis, a form of cell-death linked to inflammasome activation⁷⁶. Thus, the viability of macrophages upon 24 h incubation with CsgA filaments was probed by measuring metabolic activity and by means of the LIVE/DEAD assay. Resazurin reduction revealed that CsgA-based filaments were fully cytocompatible (FIG. 7C). Additionally, as observed by fluorescence microscopy, 24 h treatment with 30 μg/mL of 3M2e-CsgA or 3M2e-R4R5 filaments did not increase the number of cells with loss of plasma membrane integrity (dead cells), nor decrease the number of cells having intracellular esterase activity (live cells), suggestive of no toxicity (FIG. 7D). Taken together, these results indicate that the capacity of the 3M2e-CsgA and 3M2e-R4R5 nanofilaments activate the innate immunity in vitro is similar to that of the unmodified CsgA assemblies.

### Example 4: CsgA-Based Nanofilaments are Efficiently Uptaken by APCs and Induce Maturation of Dendritic Cells

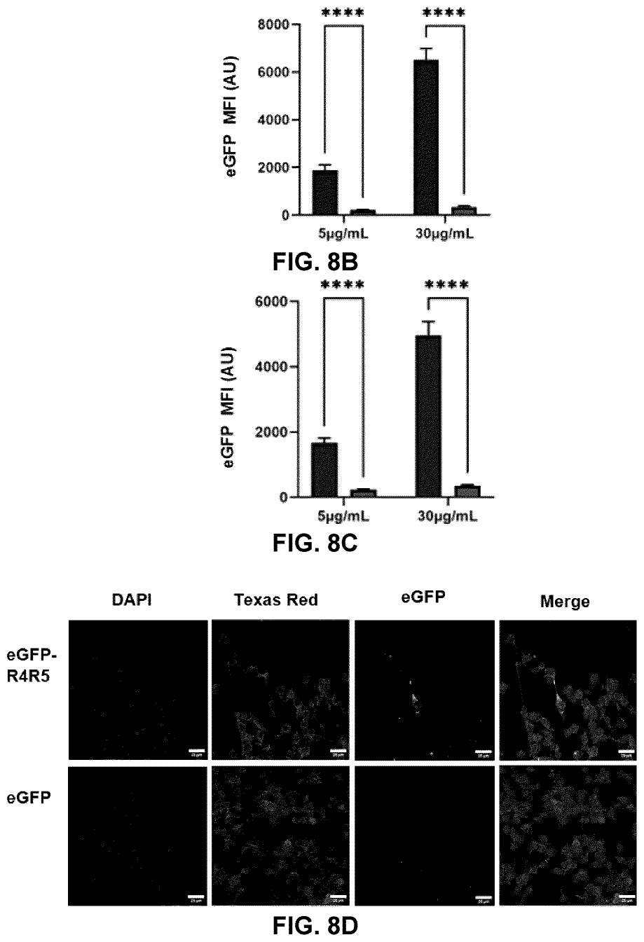

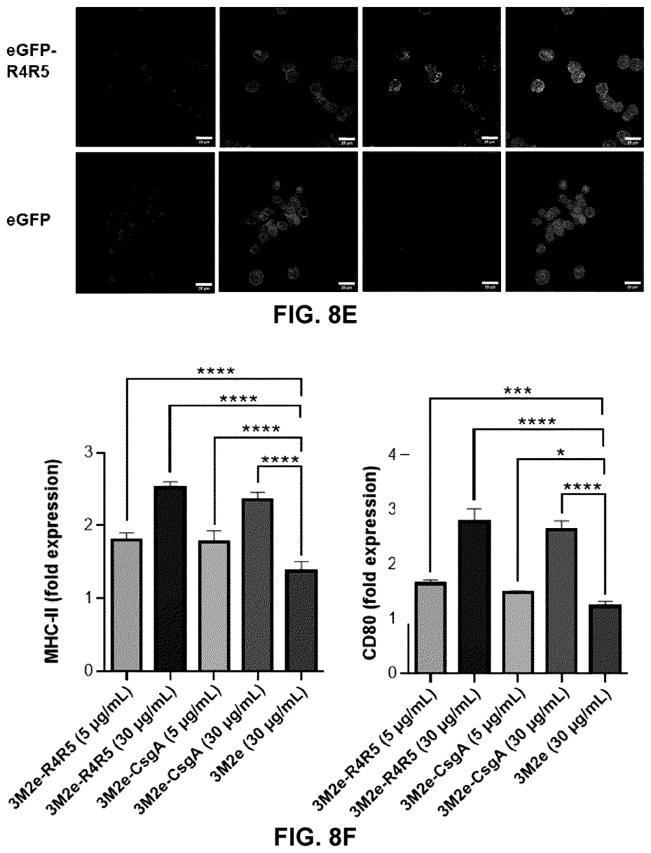

The cellular uptake of antigens by APCs is an important step for the presentation of antigen-derived peptides on major histocompatibility complex (MHC) and the induction of antigen-specific adaptive immune response. To observe internalization of CsgA-based filaments, the 3M2e antigen was replaced with an enhanced green fluorescent protein (eGFP) for confocal fluorescence microscopy and flow cytometry analysis. Bearing in mind that the eGFP in fusion with full-length CsgA, or R4R5, did not refold properly following treatment with a high concentration of denaturants needed for purification, the eGFP-CsgA and eGFP-R4R5 chimeric proteins needed to be purified under native conditions. However, under these conditions, full-length CsgA conjugated to eGFP readily aggregated into large amorphous aggregates in the purification process, precluding analysis with well-defined fibrils. Accordingly, only the eGFP-R4R5 chimeric protein was used to study internalization by APCs. The self-assembly of the fragment R4R5 tolerated the addition of a large protein as eGFP (FIG. 8A), suggestive of robust self-recognition properties. This offers the possibility to conjugate large conformational antigens to R4R5, such as viral glycoproteins. DC2.4 dendritic cells and J774.A1 macrophages were respectively incubated for 3 h with 5 μg/mL and 30 μg/mL of eGFP-R4R5 filaments, or soluble eGFP, before analysis by confocal fluorescence microscopy and flow cytometry to quantify and visualize the internalized eGFP. As observed by flow cytometry, conjugation of eGFP to R4R5 filaments drastically increased its internalization by both macrophages and dendritic cells (FIGS. 8B and 8C).

Confocal microscopy images further exposed that the eGFP-R4R5 filaments were avidly internalized by both macrophages and dendritic cells (FIGS. 8D and 8E). Following antigen uptake and TLR activation, dendritic cells begin to mature and the expression of MHC and co-stimulatory molecules like CD80 is upregulated⁷⁸. As the CsgA-based filaments activate the TLR2-TLR1 (FIG. 7A), it was hypothesized that uptake by DCs will induce the upregulation of maturation markers. To verify this, cells were treated with 3M2e-CsgA or 3M2e-R4R5 fibrils for 16 h and the expression of MHC-II and the co-stimulatory molecule CD80 was evaluated by immunohistochemistry. The results showed an increase of more than two-fold for CD80 and MHC-II expression upon 16 h treatment with both filaments relative to vehicle control (FIG. 8F). Taken together, these results show that CsgA-based filaments are efficiently internalized by APCs and induce the maturation of DCs, which is an important step for T cell activation and the induction of antigen-specific immune response.

### Example 5: Intramuscular Administration of 3M2e-R4R5 Nanofilaments Induces a Robust Anti-M2e Specific Humoral Immune Response and Protects Mice Against Experimental Infection

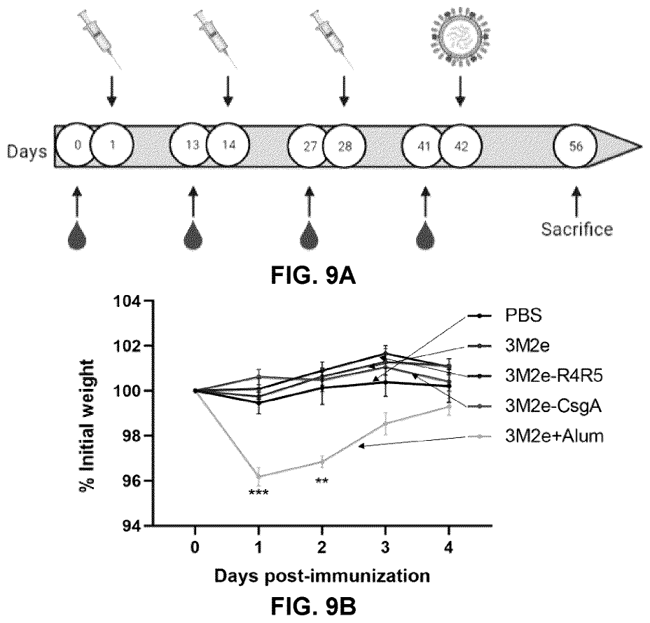

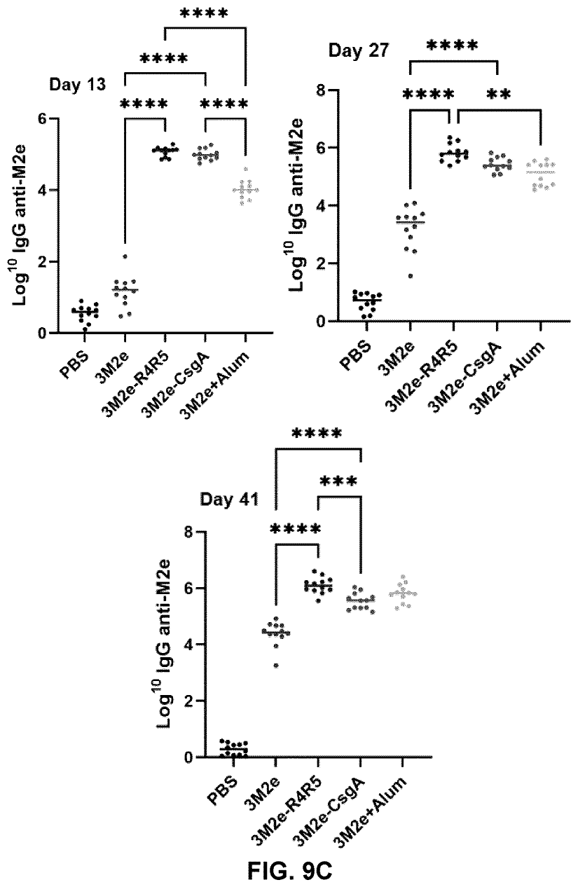

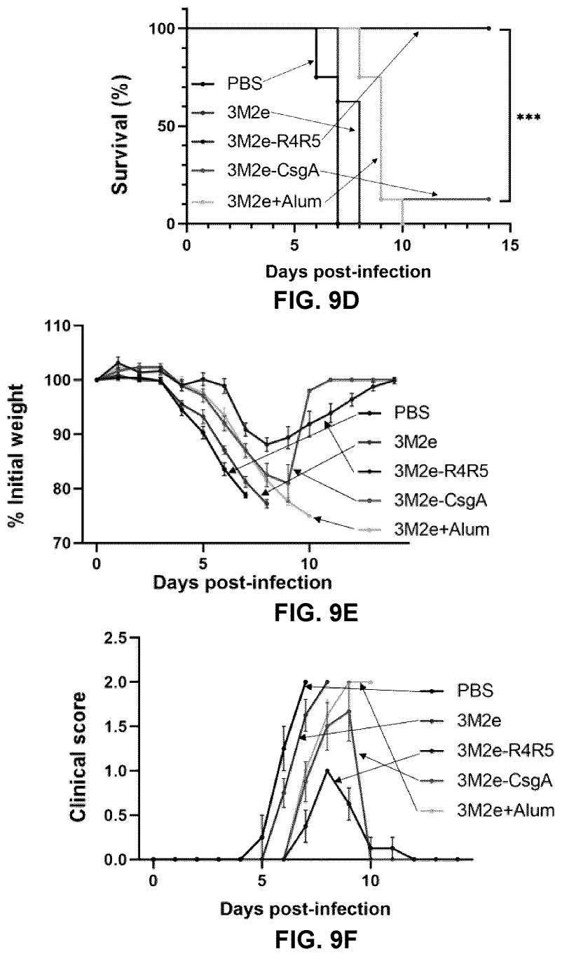

The antigen delivery and immunostimulating properties of CsgA-based nanofilaments were evaluated in vivo by immunizing mice intramuscularly and measuring the M2e-specific humoral immune response. Mice received three doses of equimolar concentration of 3M2e (18 μg of 3M2e, 30 μg of 3M2e-R4R5, and 50 μg of 3M2e-CsgA) at 14 days intervals (FIG. 9A). Sera were collected from the saphenous vein 13 days after each immunization and the anti-M2e specific antibody response was measured by indirect ELISA. Following the initial dose, a significant decrease in weight was observed in mice that received 3M2e with aluminum salts (Alum) relative to mice that were immunized with 3M2e-filaments (FIG. 9B). Following the first immunization, mice that received 3M2e-CsgA and 3M2e-R4R5 formulations had significantly higher anti-M2e total IgGs relative to mice that received soluble 3M2e, in absence, or in combination with Alum (FIG. 5C). After the first boost, only mice that received the 3M2e-R4R5 vaccine had significantly higher antibody titers than the 3M2e+Alum immunized mice, suggesting that R4R5 induces a more potent immune response than CsgA. Following the three immunizations, mice that received 3M2e+Alum had similar anti-M2e IgG titer relative to the ones that were immunized with both platforms. Interestingly, mice that received 3M2e-R4R5 had significantly higher anti-M2e IgG relative to mice immunized with the 3M2e-CsgA vaccine. According to the observed robust humoral immune responses conferred by CsgA-based vaccines, the capacity of the formulations to protect mice from an experimental IAV challenge was evaluated. Fourteen days after the last immunization, mice were infected by intranasal instillation with 5×LD₅₀ of influenza strain A/H1N1/Puerto Rico/8/1934. Weight loss and clinical signs, including temperature, activity, and posture (Table 1), were monitored daily and mice that lost more than 20% of their weight were sacrificed and reported as dead animals. Eight to ten days post-infection (PI), mice immunized with PBS control and the soluble 3M2e proteins, with or without Alum, showed 100% mortality (FIG. 9D). The 3M2e-CsgA formulation provided no significant protection, as only 12.5% of mice survived. In sharp contrast, immunization with 3M2e-R4R5 conferred 100% survival with a mean of weight loss of approximately 10% and moderate clinical signs (FIGS. 9E and 9F). Mice that survived the infection got back to their initial weight and had no clinical score 14 days after the infection. These results highlight the efficacy of the immune protection conferred by 3M2e-R4R5 vaccines against a lethal dose of the HIN1 IAV virus in mice.

### Example 6: CsgA-Based Nanofilaments Induce a Balanced Th1/Th2 M2e-Specific Immune Response in Mice

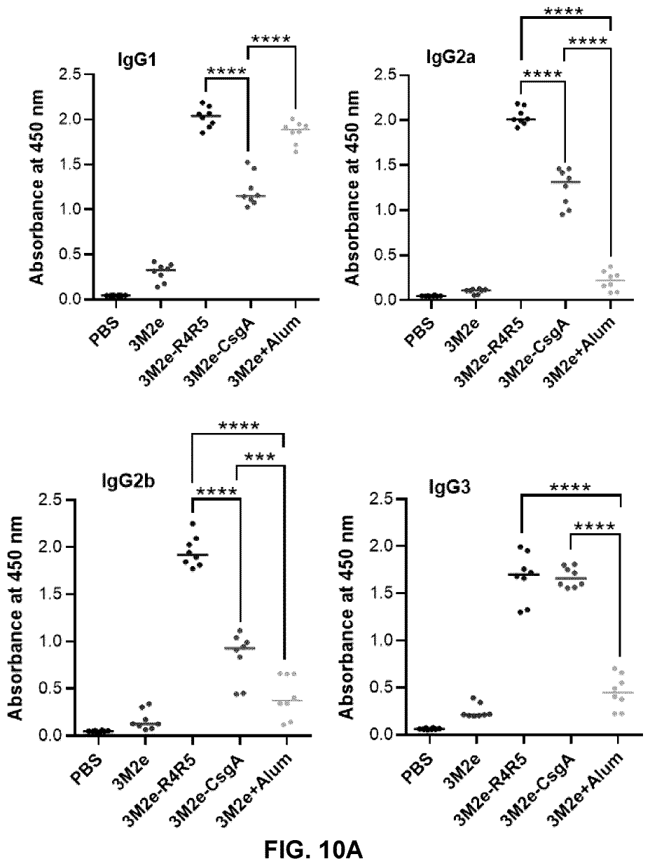

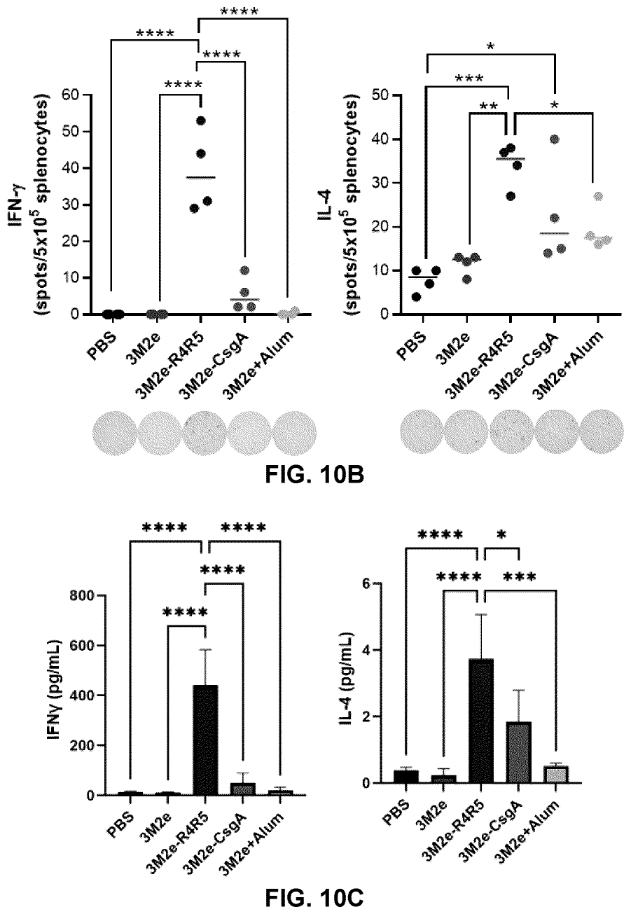

Considering the difference in protection conferred by the truncated CsgA platform (R4R5) relative to the full-length filament, the polarization of the M2e-specific immune response was analyzed to provide insight on the molecular basis of protection. In the context of vaccination against respiratory viruses, Th1 polarization or a mixed Th1/Th2 response is associated with tissue protection, while a Th2 polarization is linked to enhanced respiratory disease (ERD) pathologies⁷⁹⁻⁸¹. To assess the polarization of the M2e-specific immune response, the IgG isotypes present in the sera of mice 14 days after the third dose were assessed. IgG isotyping is an indicator of T helper polarization and IgG1 are associated with a Th2 response while IgG2a are related to a Th1 polarization⁸². Furthermore, isotypes have different efficacy at mediating Fc effector function, which is important for M2e-mediated control of IAV infection. Mice immunized with the 3M2e-R4R5 vaccine showed the highest level of IgG1, whereas mice that received 3M2e-CsgA and 3M2e+Alum had a comparable level (FIG. 10A). IgG1 from 3M2e-immunized mice without Alum were barely detectable at this dilution. Levels of IgG2a and IgG2b were significantly higher for mice immunized with 3M2e-R4R5 relative to mice that received 3M2e-CsgA and soluble 3M2e+Alum. This observation potentially explains the protection conferred by 3M2e-R4R5 since IgG2a is the most efficient subclass of IgG-mediated Fc effector function in mice⁸³. Levels of IgG3 were similar between the 3M2e-CsgA and the 3M2e-R4R5 groups, while being significantly higher relative to 3M2e+Alum. Mice immunized with 3M2e+Alum showed low levels of Ig2a, IgG2b, and IgG384. In fact, while the 3M2e-displaying CsgA-based filaments induced a mixed response in IgG isotypes, indicative of a Th1/Th2 response, 3M2e+Alum only induced IgG1 which is typical of a Th2 response⁸⁵. To further evaluate this hypothesis, the cellular immune response of immunized mice (n=4 per group) was analyzed by collecting the spleens seven days after the last boost and by restimulating the isolated splenocytes with synthetic M2e peptide. The secretion of interferon gamma (IFNγ) and interleukin-4 (IL-4), which are respectively associated with Th1 and Th2 responses, was assessed by ELISPOT. Upon ex vivo restimulation with the M2e peptide, robust IFNγ and IL-4 responses were detected in splenocytes isolated from mice immunized with the 3M2e-R4R5 formulation (FIG. 10B). Splenocytes from mice immunized with 3M2e-CsgA only showed a modest IL-4 response while the IFNγ production was not significantly higher than PBS-immunized mice. No significant cellular immune response was detected from mice that received soluble 3M2e+Alum. The cytokine levels in the supernatant of ex vivo M2e-stimulated splenocytes were also measured by sandwich ELISA. Splenocytes of 3M2e-R4R5 immunized mice showed the highest secretion of IFNγ and IL-4 (FIG. 10C). Intriguingly, the secretion of IL-4 from splenocytes of mice inoculated with 3M2e-CsgA was not significantly higher than the PBS-immunized mice. No significant secretion of IFNγ or IL-4 was detected from M2e-stimulated splenocytes isolated from mice immunized with the 3M2e peptide, with or without Alum. The stronger cellular immune response directed toward M2e in 3M2e-R4R5 immunized mice may explain why these mice were completely protected upon experimental infection, relative to the 3M2e-CsgA immunized mice. It was reported that CD4 T cells are necessary for protection conferred by universal influenza vaccine in mice⁸⁶. Furthermore, the presence of M2e-specific IFNγ production could facilitate macrophages and NK cells recruitment and the elimination of infected cells⁸⁷⁻⁸⁹ and limit viral entry by diminishing sialic acid, the receptor for IAV cell entry, clustering at the surface of airway epithelial cells⁹⁰.

Taken together, these results highlight the potential of 3M2e-R4R5 filaments to induce a strong and mixed Th1/Th2 M2e-specific immune response.

### Example 7: CsgA-Based Nanofilaments do not Induce Adverse Overactivation of Inflammatory Response

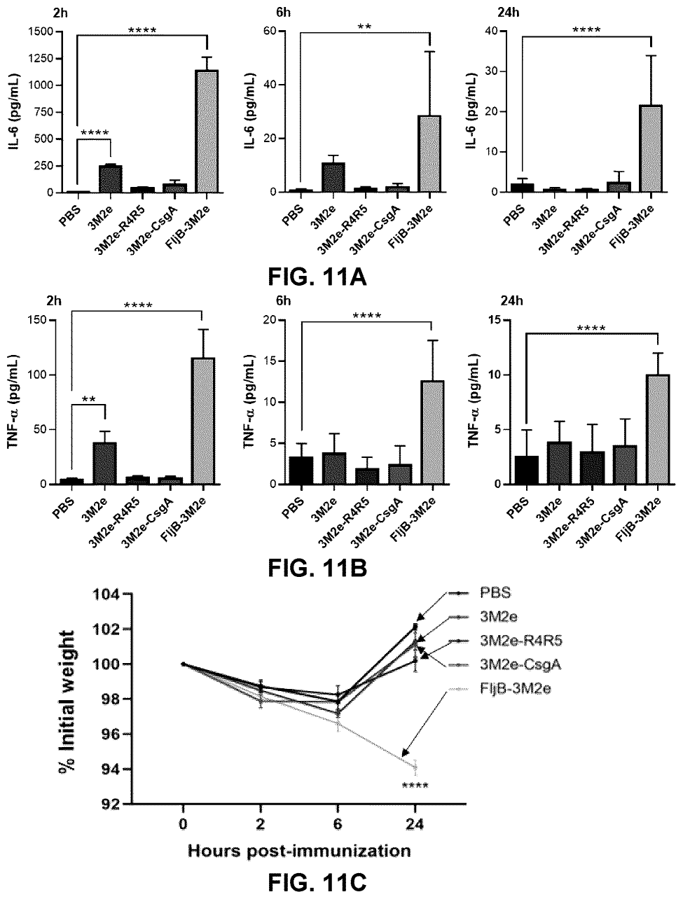

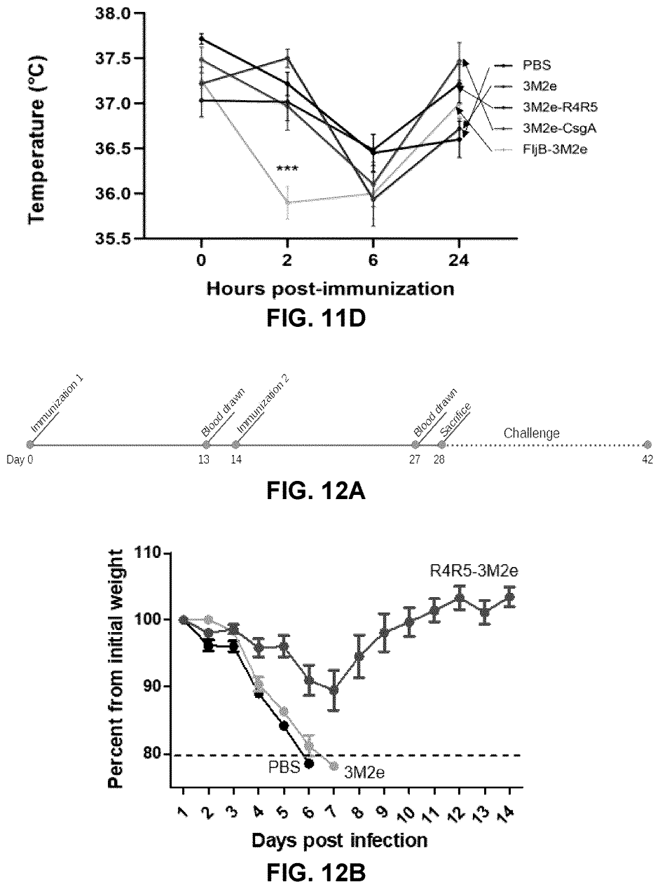

The use of pathogen-associated molecular patterns (PAMPs) as vaccine adjuvants can be associated with an overstimulation of the immune system and can lead to sustained systemic inflammation and off-target toxicity^{30,95,96}. For example, the bacterial protein flagellin (FlgB), a TLR5 agonist that has been clinically evaluated as a vaccine adjuvant, has induced severe pro-inflammatory symptoms in immunized individuals associated with elevated inflammatory markers in the blood⁹⁷. Thus, the levels of the pro-inflammatory cytokines IL-6 and tumor necrosis factor α (TNFα) in the serum was measured 2, 6, and 24 h after intraperitoneal (IP) inoculation. This route of administration was chosen since it favors systemic exposure⁹⁸ to antigenic materials. Two to 24 h following IP inoculation with CsgA-based filaments, the levels of IL-6 and TNFα in the sera were similar to the cytokine levels from control mice inoculated with PBS (FIGS. 11A, 11B). Mice inoculated with FljB-3M2e, which was expected to induce a significant state of inflammation, had significantly higher levels of both pro-inflammatory cytokines for all timepoints, which did not return to baseline 24 h after IP inoculation. Surprisingly, inoculation with the soluble 3M2e induced significant levels of IL-6 and TNF-α 2 h after IP administration. Considering that the 3M2e soluble protein has a low molecular weight and can readily diffuse in the vasculature, the elevated levels of cytokines likely reflect systemic dispersion 19.20. In contrast, conjugation of 3M2e on CsgA and R4R5, appears to preclude the passive diffusion of antigenic materials within the blood stream upon inoculation, similar to the depot effect. IL-1β levels in the mice sera were also monitored but were too low to be detectable by ELISA. The rectal temperature and weight loss were also monitored at the same timepoints. Mice that received FljB-3M2e had a significant decrease of body temperature at 2 h post-inoculation and a significant weight loss was observed 24 h after inoculation (FIGS. 11C and 11D). In contrast, no significant weight loss and decrease of body temperature were observed in mice inoculated with soluble 3M2e, 3M2e-CsgA and 3M2e-R4R5 or PBS (control group). Altogether, the results suggest that CsgA-based vaccine formulations are safe and innocuous.

### Example 8: Intranasal Administration of 3M2e-R4R5 Nanofilaments Protects Mice Against Experimental Influenza Infection

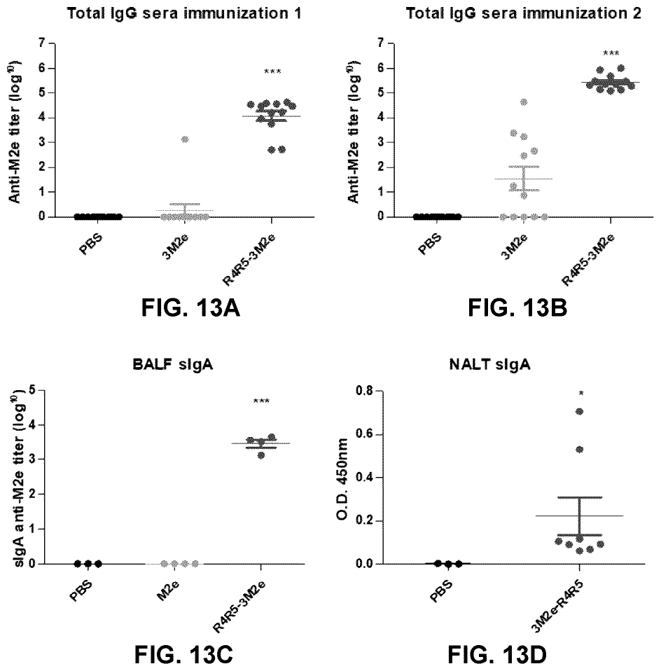

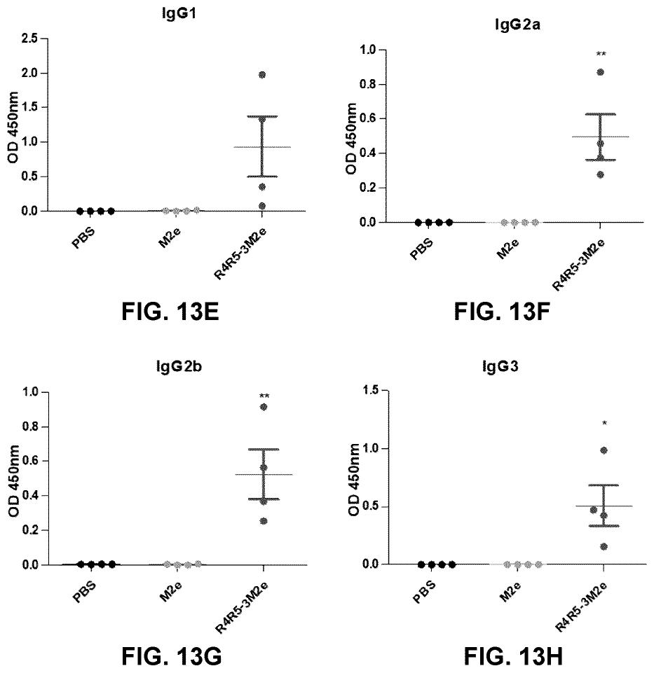

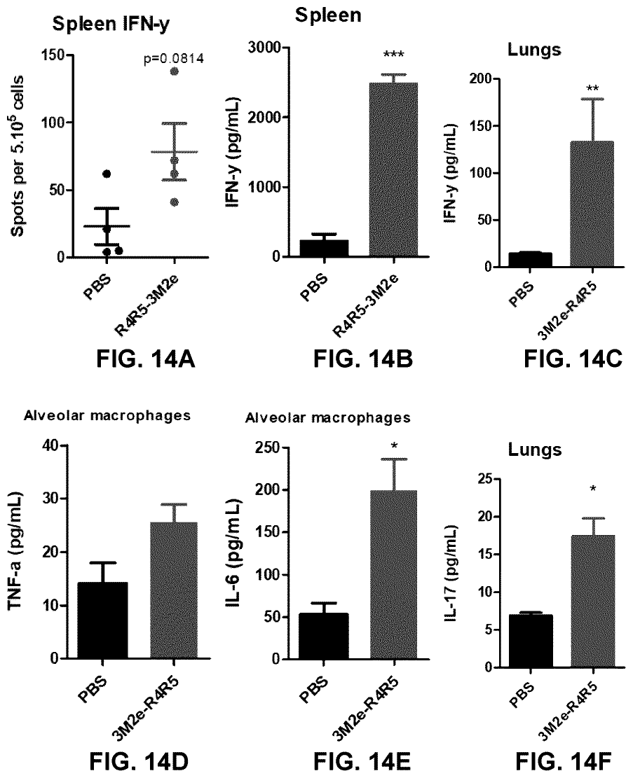

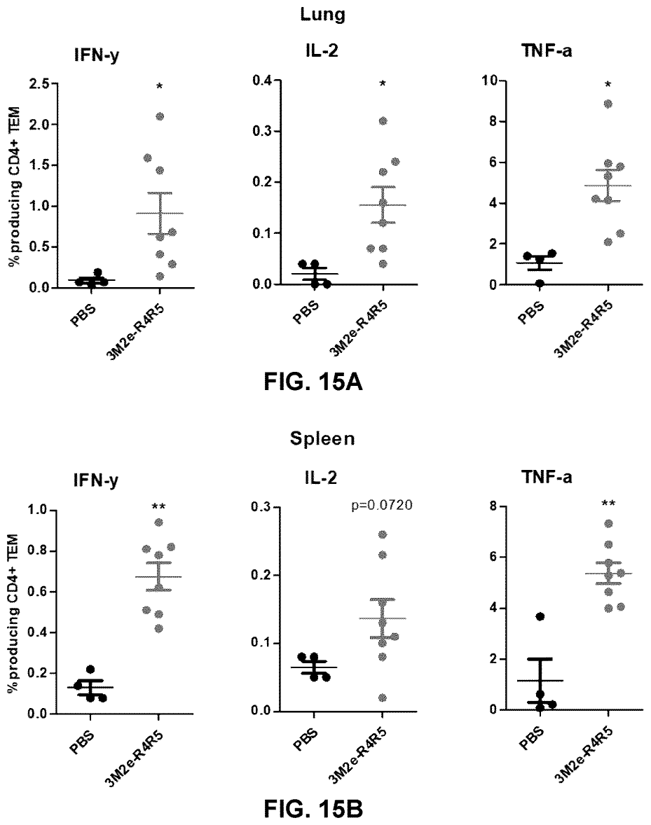

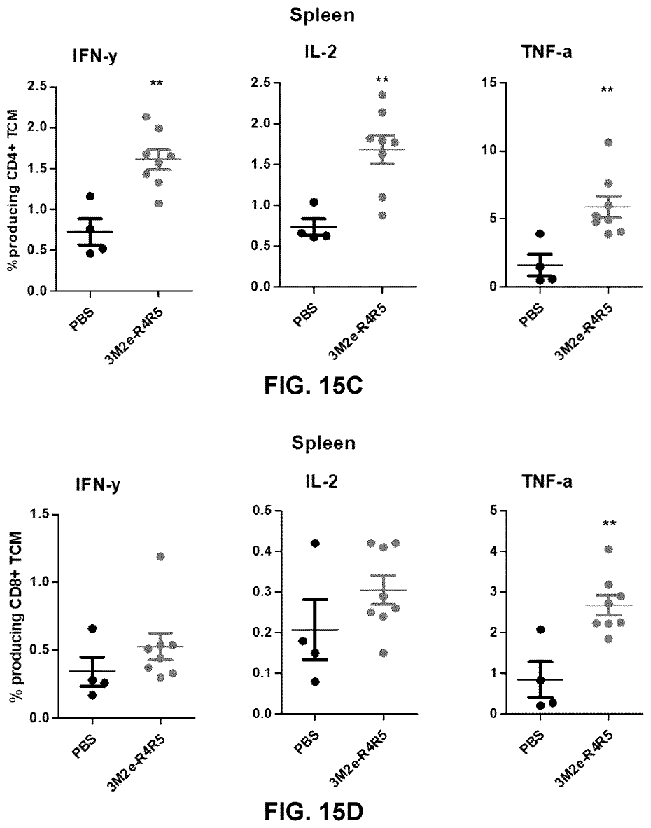

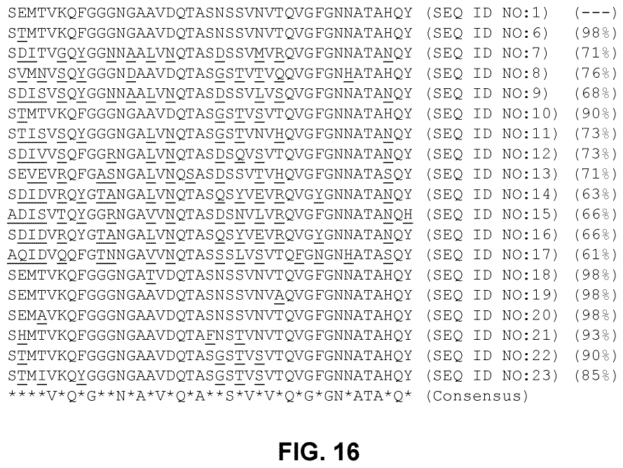

The antigen delivery and immunostimulating properties of CsgA-based nanofilaments were evaluated in vivo by immunizing mice intranasally according to the schedule depicted in FIG. 12A, and assessing weight, clinical score, survival, and induction of an M2e-specific immune response. Following infection, a transient decrease in weight was observed in mice that received the 3M2e-R4R5 conjugate, whereas a sharp weight loss occurred in animals treated with PBS (vehicle) or unconjugated 3Me2 (FIG. 12B). Similarly, whereas only a modest and transient increase in the clinical score was observed in mice immunized with 3M2e-R4R5, a sharp increase in the clinical score starting at day 4 post-infection occurred in PBS- and 3Me2-treated animals (FIG. 12C). Consistent with these results, all animals immunized with PBS and 3Me2 were dead at day 7 post-infection, whereas more than 80% of the animals immunized with the 3M2e-R4R5 formulation were alive at day 14 post-infection. Mice that received 3M2e-R4R5 formulation had significantly higher anti-M2e total IgGs, IgG2, IgG2b, IgG3, BALF sIgA, and NALT sIgA relative to mice that received soluble 3M2e or PBS (FIGS. 13A-13F). The 3M2e-R4R5 formulation also induced a strong cellular immune response, as evidenced by the increase in cytokine production (IFNγ, IL-6, TNFα, IL-2, and IL-17) by immune cells relative to control mice treated with PBS (FIGS. 14A-14F and 15A-15D).

These results demonstrate that, similar to intramuscular immunization, intranasal immunization of the 3M2e-R4R5 conjugate induces a potent immune response that confers protection against lethal influenza infection.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Francis, M. J., Recent Advances in Vaccine Technologies. Vet Clin North Am Small Anim Pract 2018, 48 (2), 231-241.
2. Orenstein, W. A.; Ahmed, R., Simply put: Vaccination saves lives. Proc Natl Acad Sci USA 2017, 114 (16), 4031-4033.
3. Beyer, W. E.; Palache, A. M.; de Jong, J. C.; Osterhaus, A. D., Cold-adapted live influenza vaccine versus inactivated vaccine: systemic vaccine reactions, local and systemic antibody response, and vaccine efficacy. A meta-analysis. Vaccine 2002, 20 (9-10), 1340-53.
4. Hanley, K. A., The double-edged sword: How evolution can make or break a live-attenuated virus vaccine. Evolution (N Y) 2011, 4 (4), 635-643.
5. Karch, C. P.; Burkhard, P., Vaccine technologies: From whole organisms to rationally designed protein assemblies. Biochem Pharmacol 2016, 120, 1-14.
6. Pulendran, B.; P, S. A.; O'Hagan, D. T., Emerging concepts in the science of vaccine adjuvants. Nat Rev Drug Discov 2021, 20 (6), 454-475.
7. Roth, G. A.; Picece, V.; Ou, B. S.; Luo, W.; Pulendran, B.; Appel, E. A., Designing spatial and temporal control of vaccine responses. Nat Rev Mater 2021, 7 (3): 174-195.
8. Marcandalli, J.; Fiala, B.; Ols, S.; Perotti, M.; de van der Schueren, W.; Snijder, J.; Hodge, E.; Benhaim, M.; Ravichandran, R.; Carter, L.; Sheffler, W.; Brunner, L.; Lawrenz, M.; Dubois, P.; Lanzavecchia, A.; Sallusto, F.; Lee, K. K.; Veesler, D.; Correnti, C. E.; Stewart, L. J.; Baker, D.; Lore, K.; Perez, L.; King, N. P., Induction of Potent Neutralizing Antibody Responses by a Designed Protein Nanoparticle Vaccine for Respiratory Syncytial Virus. Cell 2019, 176 (6), 1420-1431 e17.
9. Foged, C.; Brodin, B.; Frokjaer, S.; Sundblad, A., Particle size and surface charge affect particle uptake by human dendritic cells in an in vitro model. Int J Pharm 2005, 298 (2), 315-22.
10. Manolova, V.; Flace, A.; Bauer, M.; Schwarz, K.; Saudan, P.; Bachmann, M. F., Nanoparticles target distinct dendritic cell populations according to their size. Eur J Immunol 2008, 38 (5), 1404-13.
11. Moon, J. J.; Suh, H.; Li, A. V.; Ockenhouse, C. F.; Yadava, A.; Irvine, D. J., Enhancing humoral responses to a malaria antigen with nanoparticle vaccines that expand Tfh cells and promote germinal center induction. Proc Natl Acad Sci USA 2012, 109 (4), 1080-5.
12. Petithory, T.; Pieuchot, L.; Josien, L.; Ponche, A.; Anselme, K.; Vonna, L., Size-Dependent Internalization Efficiency of Macrophages from Adsorbed Nanoparticle-Based Monolayers. Nanomaterials (Basel) 2021, 11 (8).
13. Snapper, C. M., Distinct Immunologic Properties of Soluble Versus Particulate Antigens. Front Immunol 2018, 9, 598.
14. Yus, C.; Irusta, S.; Sebastian, V.; Arruebo, M., Controlling Particle Size and Release Kinetics in the Sustained Delivery of Oral Antibiotics Using pH-Independent Mucoadhesive Polymers. Mol Pharm 2020, 17 (9), 3314-3327.
15. Zhang, Y. N.; Lazarovits, J.; Poon, W.; Ouyang, B.; Nguyen, L. N. M.; Kingston, B. R.; Chan, W. C. W., Nanoparticle Size Influences Antigen Retention and Presentation in Lymph Node Follicles for Humoral Immunity. Nano Lett 2019, 19 (10), 7226-7235.
16. Fries, C. N.; Curvino, E. J.; Chen, J. L.; Permar, S. R.; Fouda, G. G.; Collier, J. H., Advances in nanomaterial vaccine strategies to address infectious diseases impacting global health. Nat Nanotechnol 2021, 16 (4), 1-14.
17. Puffer, E. B.; Pontrello, J. K.; Hollenbeck, J. J.; Kink, J. A.; Kiessling, L. L., Activating B cell signaling with defined multivalent ligands. ACS Chem Biol 2007, 2 (4), 252-62.
18. Irvine, D. J.; Aung, A.; Silva, M., Controlling timing and location in vaccines. Adv Drug Deliv Rev 2020, 158, 91-115.
19. Miller, N. E.; Michel, C. C.; Nanjee, M. N.; Olszewski, W. L.; Miller, I. P.; Hazell, M.; Olivecrona, G.; Sutton, P.; Humphreys, S. M.; Frayn, K. N., Secretion of adipokines by human adipose tissue in vivo: partitioning between capillary and lymphatic transport. Am J Physiol Endocrinol Metab 2011, 301 (4), E659-67.
20. Supersaxo, A.; Hein, W. R.; Steffen, H., Effect of molecular weight on the lymphatic absorption of water-soluble compounds following subcutaneous administration. Pharm Res 1990, 7 (2), 167-9.
21. Al-Halifa, S.; Gauthier, L.; Arpin, D.; Bourgault, S.; Archambault, D., Nanoparticle-Based Vaccines Against Respiratory Viruses. Front Immunol 2019, 10, 22.
22. Bachmann, M. F.; Jennings, G. T., Vaccine delivery: a matter of size, geometry, kinetics and molecular patterns. Nat Rev Immunol 2010, 10 (11), 787-96.
23. Irvine, D. J.; Swartz, M. A.; Szeto, G. L., Engineering synthetic vaccines using cues from natural immunity. Nat Mater 2013, 12 (11), 978-90.
24. Mendy, M.; Peterson, I.; Hossin, S.; Peto, T.; Jobarteh, M. L.; Jeng-Barry, A.; Sidibeh, M.; Jatta, A.; Moore, S. E.; Hall, A. J.; Whittle, H., Observational study of vaccine efficacy 24 years after the start of hepatitis B vaccination in two Gambian villages: no need for a booster dose. PLOS One 2013, 8 (3), e58029.
25. Kjaer, S. K.; Nygard, M.; Sundstrom, K.; Dillner, J.; Tryggvadottir, L.; Munk, C.; Berger, S.; Enerly, E.; Hortlund, M.; Agustsson, A. I.; Bjelkenkrantz, K.; Fridrich, K.; Guethmundsdottir, I.; Sorbye, S. W.; Bautista, O.; Group, T.; Luxembourg, A.; Marshall, J. B.; Radley, D.; Yang, Y. S.; Badshah, C.; Saah, A., Final analysis of a 14-year long-term follow-up study of the effectiveness and immunogenicity of the quadrivalent human papillomavirus vaccine in women from four nordic countries. EClinicalMedicine 2020, 23, 100401.
26. Carmen, J. M.; Shrivastava, S.; Lu, Z.; Anderson, A.; Morrison, E. B.; Sankhala, R. S.; Chen, W. H.; Chang, W. C.; Bolton, J. S.; Matyas, G. R.; Michael, N. L.; Joyce, M. G.; Modjarrad, K.; Currier, J. R.; Bergmann-Leitner, E.; Malloy, A. M. W.; Rao, M., SARS-COV-2 ferritin nanoparticle vaccine induces robust innate immune activity driving polyfunctional spike-specific T cell responses. NPJ Vaccines 2021, 6 (1), 151.
27. Garg, H.; Mehmetoglu-Gurbuz, T.; Joshi, A., Virus Like Particles (VLP) as multivalent vaccine candidate against Chikungunya, Japanese Encephalitis, Yellow Fever and Zika Virus. Sci Rep 2020, 10 (1), 4017.
28. Roldao, A.; Mellado, M. C.; Castilho, L. R.; Carrondo, M. J.; Alves, P. M., Virus-like particles in vaccine development. Expert Rev Vaccines 2010, 9 (10), 1149-76.
29. Zhang, B.; Chao, C. W.; Tsybovsky, Y.; Abiona, O. M.; Hutchinson, G. B.; Moliva, J. I.; Olia, A. S.; Pegu, A.; Phung, E.; Stewart-Jones, G. B. E.; Verardi, R.; Wang, L.; Wang, S.; Werner, A.; Yang, E. S.; Yap, C.; Zhou, T.; Mascola, J. R.; Sullivan, N. J.; Graham, B. S.; Corbett, K. S.; Kwong, P. D., A platform incorporating trimeric antigens into self-assembling nanoparticles reveals SARS-COV-2-spike nanoparticles to elicit substantially higher neutralizing responses than spike alone. Sci Rep 2020, 10 (1), 18149.
30. Vasilakos, J. P.; Tomai, M. A., The use of Toll-like receptor 7/8 agonists as vaccine adjuvants. Expert Rev Vaccines 2013, 12 (7), 809-19.
31. Barnhart, M. M.; Chapman, M. R., Curli biogenesis and function. Annu Rev Microbiol 2006, 60, 131-47.
32. McCrate, O. A.; Zhou, X.; Reichhardt, C.; Cegelski, L., Sum of the parts: composition and architecture of the bacterial extracellular matrix. J Mol Biol 2013, 425 (22), 4286-94.
33. Pawar, D. M.; Rossman, M. L.; Chen, J., Role of curli fimbriae in mediating the cells of enterohaemorrhagic Escherichia coli to attach to abiotic surfaces. J Appl Microbiol 2005, 99 (2), 418-25.
34. Gophna, U.; Oelschlaeger, T. A.; Hacker, J.; Ron, E. Z., Role of fibronectin in curli-mediated internalization. FEMS Microbiol Lett 2002, 212 (1), 55-8.
35. Olsen, A.; Arnqvist, A.; Hammar, M.; Sukupolvi, S.; Normark, S., The RpoS sigma factor relieves H-NS-mediated transcriptional repression of csgA, the subunit gene of fibronectin-binding curli in Escherichia coli. Mol Microbiol 1993, 7 (4), 523-36.
36. Evans, M. L.; Chapman, M. R., Curli biogenesis: order out of disorder. Biochim Biophys Acta 2014, 1843 (8), 1551-8.
37. Wang, X.; Chapman, M. R., Sequence determinants of bacterial amyloid formation. J Mol Biol 2008, 380 (3), 570-80.
38. Chapman, M. R.; Robinson, L. S.; Pinkner, J. S.; Roth, R.; Heuser, J.; Hammar, M.; Normark, S.; Hultgren, S. J., Role of Escherichia coli curli operons in directing amyloid fiber formation. Science 2002, 295 (5556), 851-5.
39. Dueholm, M. S.; Nielsen, S. B.; Hein, K. L.; Nissen, P.; Chapman, M.; Christiansen, G.; Nielsen, P. H.; Otzen, D. E., Fibrillation of the major curli subunit CsgA under a wide range of conditions implies a robust design of aggregation. Biochemistry 2011, 50 (39), 8281-90.
40. Wang, X.; Hammer, N. D.; Chapman, M. R., The molecular basis of functional bacterial amyloid polymerization and nucleation. J Biol Chem 2008, 283 (31), 21530-9.
41. Wang, X.; Smith, D. R.; Jones, J. W.; Chapman, M. R., In vitro polymerization of a functional Escherichia coli amyloid protein. J Biol Chem 2007, 282 (6), 3713-9.
42. Cui, M.; Qi, Q.; Gurry, T.; Zhao, T.; An, B.; Pu, J.; Gui, X.; Cheng, A. A.; Zhang, S.; Xun, D.; Becce, M.; Briatico-Vangosa, F.; Liu, C.; Lu, T. K.; Zhong, C., Modular genetic design of multi-domain functional amyloids: insights into self-assembly and functional properties. Chem Sci 2019, 10 (14), 4004-4014.
43. Zhong, C.; Gurry, T.; Cheng, A. A.; Downey, J.; Deng, Z.; Stultz, C. M.; Lu, T. K., Strong underwater adhesives made by self-assembling multi-protein nanofibres. Nat Nanotechnol 2014, 9 (10), 858-66.
44. Bian, Z.; Brauner, A.; Li, Y.; Normark, S., Expression of and cytokine activation by Escherichia coli curli fibers in human sepsis. J Infect Dis 2000, 181 (2), 602-12.
45. Kai-Larsen, Y.; Luthje, P.; Chromek, M.; Peters, V.; Wang, X.; Holm, A.; Kadas, L.; Hedlund, K. O.; Johansson, J.; Chapman, M. R.; Jacobson, S. H.; Romling, U.; Agerberth, B.; Brauner, A., Uropathogenic Escherichia coli modulates immune responses and its curli fimbriae interact with the antimicrobial peptide LL-37. PLOS Pathog 2010, 6 (7), e1001010.
46. Tursi, S. A.; Tukel, C., Curli-Containing Enteric Biofilms Inside and Out: Matrix Composition, Immune Recognition, and Disease Implications. Microbiol Mol Biol Rev 2018, 82 (4).
47. Rapsinski, G. J.; Wynosky-Dolfi, M. A.; Oppong, G. O.; Tursi, S. A.; Wilson, R. P.; Brodsky, I. E.; Tukel, C., Toll-like receptor 2 and NLRP3 cooperate to recognize a functional bacterial amyloid, curli. Infect Immun 2015, 83 (2), 693-701.
48. Tukel, C.; Nishimori, J. H.; Wilson, R. P.; Winter, M. G.; Keestra, A. M.; van Putten, J. P.; Baumler, A. J., Toll-like receptors 1 and 2 cooperatively mediate immune responses to curli, a common amyloid from enterobacterial biofilms. Cell Microbiol 2010, 12 (10), 1495-505.
49. Tukel, C.; Raffatellu, M.; Humphries, A. D.; Wilson, R. P.; Andrews-Polymenis, H. L.; Gull, T.; Figueiredo, J. F.; Wong, M. H.; Michelsen, K. S.; Akcelik, M.; Adams, L. G.; Baumler, A. J., CsgA is a pathogen-associated molecular pattern of Salmonella enterica serotype Typhimurium that is recognized by Toll-like receptor 2. Mol Microbiol 2005, 58 (1), 289-304.
50. Tukel, C.; Wilson, R. P.; Nishimori, J. H.; Pezeshki, M.; Chromy, B. A.; Baumler, A. J., Responses to amyloids of microbial and host origin are mediated through toll-like receptor 2. Cell Host Microbe 2009, 6 (1), 45-53.
51. Al-Halifa, S.; Zottig, X.; Babych, M.; Cote-Cyr, M.; Bourgault, S.; Archambault, D., Harnessing the Activation of Toll-Like Receptor 2/6 by Self-Assembled Cross-beta Fibrils to Design Adjuvanted Nanovaccines. Nanomaterials (Basel) 2020, 10 (10).
52. Chen, J.; Pompano, R. R.; Santiago, F. W.; Maillat, L.; Sciammas, R.; Sun, T.; Han, H.; Topham, D. J.; Chong, A. S.; Collier, J. H., The use of self-adjuvanting nanofiber vaccines to elicit high-affinity B cell responses to peptide antigens without inflammation. Biomaterials 2013, 34 (34), 8776-85.
53. Rudra, J. S.; Tian, Y. F.; Jung, J. P.; Collier, J. H., A self-assembling peptide acting as an immune adjuvant. Proc Natl Acad Sci USA 2010, 107 (2), 622-7.
54. Zottig, X.; Al-Halifa, S.; Cote-Cyr, M.; Calzas, C.; Le Goffic, R.; Chevalier, C.; Archambault, D.; Bourgault, S., Self-assembled peptide nanorod vaccine confers protection against influenza A virus. Biomaterials 2021, 269, 120672.
55. Nachbagauer, R.; Palese, P., Is a Universal Influenza Virus Vaccine Possible? Annu Rev Med 2020, 71, 315-327.
56. El Bakkouri, K.; Descamps, F.; De Filette, M.; Smet, A.; Festjens, E.; Birkett, A.; Van Rooijen, N.; Verbeek, S.; Fiers, W.; Saelens, X., Universal vaccine based on ectodomain of matrix protein 2 of influenza A: Fc receptors and alveolar macrophages mediate protection. J Immunol 2011, 186 (2), 1022-31.
57. Jegerlehner, A.; Schmitz, N.; Storni, T.; Bachmann, M. F., Influenza A vaccine based on the extracellular domain of M2: weak protection mediated via antibody-dependent NK cell activity. J Immunol 2004, 172 (9), 5598-605.
58. Simhadri, V. R.; Dimitrova, M.; Mariano, J. L.; Zenarruzabeitia, O.; Zhong, W.; Ozawa, T.; Muraguchi, A.; Kishi, H.; Eichelberger, M. C.; Borrego, F., A Human Anti-M2 Antibody Mediates Antibody-Dependent Cell-Mediated Cytotoxicity (ADCC) and Cytokine Secretion by Resting and Cytokine-Preactivated Natural Killer (NK) Cells. PLOS One 2015, 10 (4), e0124677.
59. Nguyen, P. T.; Zottig, X.; Sebastiao, M.; Arnold, A. A.; Marcotte, I.; Bourgault, S., Identification of transmissible proteotoxic oligomer-like fibrils that expand conformational diversity of amyloid assemblies. Commun Biol 2021, 4 (1), 939.
60. Zottig, X.; Al-Halifa, S.; Babych, M.; Quittot, N.; Archambault, D.; Bourgault, S., Guiding the Morphology of Amyloid Assemblies by Electrostatic Capping: from Polymorphic Twisted Fibrils to Uniform Nanorods. Small 2019, 15 (33), e1901806.
61. Nilsson, M. R., Techniques to study amyloid fibril formation in vitro. Methods 2004, 34 (1), 151-60.
62. Nicoud, L.; Lattuada, M.; Lazzari, S.; Morbidelli, M., Viscosity scaling in concentrated dispersions and its impact on colloidal aggregation. Phys Chem Chem Phys 2015, 17 (37), 24392-402.
63. Saito, S.; Ainai, A.; Suzuki, T.; Harada, N.; Ami, Y.; Yuki, Y.; Takeyama, H.; Kiyono, H.; Tsukada, H.; Hasegawa, H., The effect of mucoadhesive excipient on the nasal retention time of and the antibody responses induced by an intranasal influenza vaccine. Vaccine 2016, 34 (9), 1201-7.
64. Miquel-Clopes, A.; Bentley, E. G.; Stewart, J. P.; Carding, S. R., Mucosal vaccines and technology. Clin Exp Immunol 2019, 196 (2), 205-214.
65. Iwasaki, A.; Medzhitov, R., Toll-like receptor control of the adaptive immune responses. Nat Immunol 2004, 5 (10), 987-95.
66 Medzhitov, R., Toll-like receptors and innate immunity. Nat Rev Immunol 2001, 1 (2), 135-45.
67 Takeuchi, O.; Akira, S., Pattern recognition receptors and inflammation. Cell 2010, 140 (6), 805-20.
68. Basto, A. P.; Leitao, A., Targeting TLR2 for vaccine development. J Immunol Res 2014, 2014, 619410.
69. Kumar, S.; Sunagar, R.; Gosselin, E., Bacterial Protein Toll-Like-Receptor Agonists: A Novel Perspective on Vaccine Adjuvants. Front Immunol 2019, 10, 1144.
70. Kaur, A.; Kanwar, R.; Kaushik, D.; Sakala, I. G.; Honda-Okubo, Y.; Petrovsky, N.; Salunke, D. B.; Mehta, S. K., Combined delivery of TLR2 and TLR7 agonists by Nanostructured lipid carriers induces potent vaccine adjuvant activity in mice. Int J Pharm 2022, 613, 121378.
71. Caproni, E.; Tritto, E.; Cortese, M.; Muzzi, A.; Mosca, F.; Monaci, E.; Baudner, B.; Seubert, A.; De Gregorio, E., MF59 and Pam3CSK4 boost adaptive responses to influenza subunit vaccine through an IFN type I-independent mechanism of action. J Immunol 2012, 188 (7), 3088-98.
72. Biswas, A.; Banerjee, P.; Biswas, T., Porin of Shigella dysenteriae directly promotes toll-like receptor 2-mediated CD4+ T cell survival and effector function. Mol Immunol 2009, 46 (15), 3076-85.
73. Massari, P.; Toussi, D. N.; Tifrea, D. F.; de la Maza, L. M., Toll-like receptor 2-dependent activity of native major outer membrane protein proteosomes of Chlamydia trachomatis. Infect Immun 2013, 81 (1), 303-10.
74. Reiser, M. L.; Mosaheb, M. M.; Lisk, C.; Platt, A.; Wetzler, L. M., The TLR2 Binding Neisserial Porin PorB Enhances Antigen Presenting Cell Trafficking and Cross-presentation. Sci Rep 2017, 7 (1), 736.
75. Hirano, S.; Zhou, Q.; Furuyama, A.; Kanno, S., Differential Regulation of IL-1beta and IL-6 Release in Murine Macrophages. Inflammation 2017, 40 (6), 1933-1943.
76. Shi, J.; Zhao, Y.; Wang, K.; Shi, X.; Wang, Y.; Huang, H.; Zhuang, Y.; Cai, T.; Wang, F.; Shao, F., Cleavage of GSDMD by inflammatory caspases determines pyroptotic cell death. Nature 2015, 526 (7575), 660-5.
77. Hong, S.; Zhang, Z.; Liu, H.; Tian, M.; Zhu, X.; Zhang, Z.; Wang, W.; Zhou, X.; Zhang, F.; Ge, Q.; Zhu, B.; Tang, H.; Hua, Z.; Hou, B., B Cells Are the Dominant Antigen-Presenting Cells that Activate Naive CD4(+) T Cells upon Immunization with a Virus-Derived Nanoparticle Antigen. Immunity 2018, 49 (4), 695-708 e4.
78. Banchereau, J.; Steinman, R. M., Dendritic cells and the control of immunity. Nature 1998, 392 (6673), 245-52.
79. Eichinger, K. M.; Kosanovich, J. L.; Gidwani, S. V.; Zomback, A.; Lipp, M. A.; Perkins, T. N.; Oury, T. D.; Petrovsky, N.; Marshall, C. P.; Yondola, M. A.; Empey, K. M., Prefusion RSV F Immunization Elicits Th2-Mediated Lung Pathology in Mice When Formulated With a Th2 (but Not a Th1/Th2-Balanced) Adjuvant Despite Complete Viral Protection. Front Immunol 2020, 11, 1673.
80. Honda-Okubo, Y.; Barnard, D.; Ong, C. H.; Peng, B. H.; Tseng, C. T.; Petrovsky, N., Severe acute respiratory syndrome-associated coronavirus vaccines formulated with delta inulin adjuvants provide enhanced protection while ameliorating lung eosinophilic immunopathology. J Virol 2015, 89 (6), 2995-3007.
81. Kim, K. H.; Lee, Y. T.; Hwang, H. S.; Kwon, Y. M.; Jung, Y. J.; Lee, Y.; Lee, J. S.; Lee, Y. N.; Park, S.; Kang, S. M., Alum Adjuvant Enhances Protection against Respiratory Syncytial Virus but Exacerbates Pulmonary Inflammation by Modulating Multiple Innate and Adaptive Immune Cells. PLOS One 2015, 10 (10), e0139916.
82. Spellberg, B.; Edwards, J. E., Jr., Type 1/Type 2 immunity in infectious diseases. Clin Infect Dis 2001, 32 (1), 76-102.
83. Bruhns, P., Properties of mouse and human IgG receptors and their contribution to disease models. Blood 2012, 119 (24), 5640-9.
84. Brewer, J. M.; Conacher, M.; Hunter, C. A.; Mohrs, M.; Brombacher, F.; Alexander, J., Aluminium hydroxide adjuvant initiates strong antigen-specific Th2 responses in the absence of IL-4- or IL-13-mediated signaling. J Immunol 1999, 163 (12), 6448-54.
85. Visciano, M. L.; Tagliamonte, M.; Tornesello, M. L.; Buonaguro, F. M.; Buonaguro, L., Effects of adjuvants on IgG subclasses elicited by virus-like particles. J Transl Med 2012, 10, 4.
86. Valkenburg, S. A.; Li, O. T. W.; Li, A.; Bull, M.; Waldmann, T. A.; Perera, L. P.; Peiris, M.; Poon, L. L. M., Protection by universal influenza vaccine is mediated by memory CD4 T cells. Vaccine 2018, 36 (29), 4198-4206.
87. Aquino-Lopez, A.; Senyukov, V. V.; Vlasic, Z.; Kleinerman, E. S.; Lee, D. A., Interferon Gamma Induces Changes in Natural Killer (NK) Cell Ligand Expression and Alters NK Cell-Mediated Lysis of Pediatric Cancer Cell Lines. Front Immunol 2017, 8, 391.
88. Pak-Wittel, M. A.; Yang, L.; Sojka, D. K.; Rivenbark, J. G.; Yokoyama, W. M., Interferon-gamma mediates chemokine-dependent recruitment of natural killer cells during viral infection. Proc Natl Acad Sci USA 2013, 110 (1), E50-9.
89. Wu, C.; Xue, Y.; Wang, P.; Lin, L.; Liu, Q.; Li, N.; Xu, J.; Cao, X., IFN-gamma primes macrophage activation by increasing phosphatase and tensin homolog via downregulation of miR-3473b. J Immunol 2014, 193 (6), 3036-44.
90. Fong, C. H.; Lu, L.; Chen, L. L.; Yeung, M. L.; Zhang, A. J.; Zhao, H.; Yuen, K. Y.; To, K. K., Interferon-gamma inhibits influenza A virus cellular attachment by reducing sialic acid cluster size. iScience 2022, 25 (4), 104037.
91. Kato, Y.; Abbott, R. K.; Freeman, B. L.; Haupt, S.; Groschel, B.; Silva, M.; Menis, S.; Irvine, D. J.; Schief, W. R.; Crotty, S., Multifaceted Effects of Antigen Valency on B Cell Response Composition and Differentiation In Vivo. Immunity 2020, 53 (3), 548-563 e8.
92. Mirshahidi, S.; Ferris, L. C.; Sadegh-Nasseri, S., The magnitude of TCR engagement is a critical predictor of T cell anergy or activation. J Immunol 2004, 172 (9), 5346-55.
93. Williams, M. A.; Ravkov, E. V.; Bevan, M. J., Rapid culling of the CD4+ T cell repertoire in the transition from effector to memory. Immunity 2008, 28 (4), 533-45.
94. Batista, F. D.; Neuberger, M. S., B cells extract and present immobilized antigen: implications for affinity discrimination. EMBO J 2000, 19 (4), 513-20.
95. Tanaka, T.; Narazaki, M.; Kishimoto, T., IL-6 in inflammation, immunity, and disease. Cold Spring Harb Perspect Biol 2014, 6 (10), a016295.
96. Zhang, J. M.; An, J., Cytokines, inflammation, and pain. Int Anesthesiol Clin 2007, 45 (2), 27-37.
97. Turley, C. B.; Rupp, R. E.; Johnson, C.; Taylor, D. N.; Wolfson, J.; Tussey, L.; Kavita, U.; Stanberry, L.; Shaw, A., Safety and immunogenicity of a recombinant M2e-flagellin influenza vaccine (STF2.4xM2e) in healthy adults. Vaccine 2011, 29 (32), 5145-52.
98. Al Shoyaib, A.; Archie, S. R.; Karamyan, V. T., Intraperitoneal Route of Drug Administration: Should it Be Used in Experimental Animal Studies? Pharm Res 2019, 37 (1), 12.
99. De Filette, M.; Min Jou, W.; Birkett, A.; Lyons, K.; Schultz, B.; Tonkyro, A.; Resch, S.; Fiers, W., Universal influenza A vaccine: optimization of M2-based constructs. Virology 2005, 337 (1), 149-61.
100. Cote-Cyr, M.; Gauthier, L.; Zottig, X.; Bourgault, S.; Archambault, D., Recombinant Bacillus subtilis flagellin Hag is a potent immunostimulant with reduced proinflammatory properties compared to Salmonella enterica serovar Typhimurium FljB. Vaccine 2022, 40 (1), 11-17.

What is claimed is:

1. A conjugate comprising:
(i) a self-assembling polypeptide comprising an amino acid sequence having at least 60% identity with the sequence of the R4 and R5 domains from a Curli-specific gene A (CsgA) protein, wherein the self-assembling polypeptide is not a full-length CsgA protein; and
(ii) a heterologous immunogen conjugated to the self-assembling polypeptide.

2. The conjugate of claim 1, wherein the self-assembling polypeptide comprises or consists of an amino acid sequence of the following formula:
(SEQ ID NO: 24)
X1-X2-X3-X4-V-X5-Q-X6-G-X7-X8-N-X9-A-X10-V-X11-Q-
X12-A-X13-X14-S-X15-V-X16-V-X17-Q-X18-G-X19-G-N-
X20-A-T-A-X21-Q-X22
wherein X1 is S or A, preferably S;
X2 is E, D, T, or V; preferably E or D;
X3 is M, I, or V, preferably M or I;
X4 is T, N, S, V, E, or D, preferably T;
X5 is K, G, S, R, T, or Q, preferably K or R;
X6 is an aromatic residue, preferably F or Y;
X7 is G, A, or T, preferably G;
X8 is G, N, R, S, or A, preferably G;
X9 is G, A, or D, preferably G;
X10 is A, L, or V, preferably A;
X11 is D or N;
X12 is S or T, preferably T;
X13 is S or F, preferably S;
X14 is N, D, G, Q, or S, preferably N;
X15 is S, T, Q, Y, N or L, preferably S;
X16 is N, M, T, L, S, or E, preferably N;
X17 is T, R, Q, S, H, preferably T;
X18 is V or F, preferably V;
X19 is F, Y or N, preferably F;
X20 is N or H, preferably N;
X21 is H, N, or S, preferably H; and
X22 is Y or H, preferably Y.

3. The conjugate of claim 1 or 2, wherein the self-assembling polypeptide comprises an amino acid sequence having at least 60% with any one of the sequences of SEQ ID NOs: 1 and 6 to 17.

4. The conjugate of claim 3, wherein the self-assembling polypeptide comprises an amino acid sequence having at least 80% identity with any one of the sequences of SEQ ID NOs: 1 and 6 to 17.

5. The conjugate of claim 3, wherein the self-assembling polypeptide comprises an amino acid sequence having at least 90% identity with any one of the sequences of SEQ ID NOs: 1 and 6 to 17.

6. The conjugate of any one of claims 3 to 5, wherein the self-assembling polypeptide comprises an amino acid sequence having at least 60%, 80%, or 90% identity with the sequence of SEQ ID NO:1.

7. The conjugate of claim 6, wherein the self-assembling polypeptide comprises the amino acid sequence of SEQ ID NO:1.

8. The conjugate of any one of claims 1 to 7, wherein the self-assembling polypeptide has a length of 60, 50, or 45 amino acids or less.

9. The conjugate of any one of claims 1 to 8, wherein the immunogen is a protein from a microorganism or a peptide fragment thereof comprising at least 10 amino acids.

10. The conjugate of claim 9, wherein the immunogen is a viral protein, a bacterial protein, a fungal protein, a protozoan protein, a helminth protein, or a peptide fragment thereof.

11. The conjugate of claim 10, wherein the is a viral protein or a peptide fragment thereof.

12. The conjugate of claim 11, wherein the viral protein or peptide fragment thereof is a protein from influenza virus or a peptide fragment thereof.

13. The conjugate of claim 12, wherein the immunogen is a peptide fragment derived from the extracellular domain of the influenza M2 protein (M2e).

14. The conjugate of any one of claims 1 to 8, wherein the immunogen is a tumor-specific antigen or an allergen.

15. The construct of any one of claims 1 to 14, wherein the immunogen is a peptide fragment of 10 to 50 amino acids.

16. The conjugate of claim 15, wherein the immunogen comprises the sequence SLLTEVETPIRNEWGSRSNGSSD (SEQ ID NO:2).

17. The conjugate of claim 16, wherein the immunogen comprises three repeats of the sequence of SEQ ID NO:2.

18. The conjugate of claim 17, wherein the immunogen comprises the sequence of SEQ ID NO: 27.

19. The conjugate of any one of claims 1 to 18, further comprising a linker between the immunogen and the self-assembling polypeptide.

20. The conjugate of claim 19, wherein the linker is a peptide linker of 4 to 16 amino acids.

21. The conjugate of claim 20, wherein the linker is a peptide linker of 4 to 10 amino acids.

22. The conjugate of any one of claims 19 to 21, wherein the linker comprises glycine and/or serine residues.

23. The conjugate of claim 22, wherein the linker comprises the motif (GGGS) n, wherein n is an integer from 1 to 4.

24. The conjugate of claim 23, wherein n is 2.

25. The conjugate of any one of claims 1 to 24, wherein the immunogen is conjugated to the amino-terminal end of the self-assembling polypeptide.

26. The conjugate of any one of claims 1 to 25, wherein the conjugate assembled into nanofilaments having a diameter of about 2 to about 20 nm, or of about 5 to about 10 nm.

27. A nucleic acid encoding the conjugate of any one of claims 1 to 26.

28. A composition comprising the conjugate of any one of claims 1 to 26 or the nucleic acid of claim 27.

29. The composition of claim 28, further comprising a pharmaceutically acceptable carrier.

30. A vaccine comprising the conjugate of any one of claims 1 to 26, the nucleic acid of claim 27, or the composition of claim 28 or 29.

31. The vaccine of claim 30, which further comprises an adjuvant.

32. A method for inducing an immune response against an immunogen in a subject comprising administering to the subject an effective amount of: (i) the conjugate of any one of claims 1 to 26; (ii) the nucleic acid of claim 27; (iii) the composition of claim 28 or 29; or (iv) the vaccine of claim 30 or 31.

33. A method for preventing and/or treating a microbial infection, an autoimmune disease, an allergy or cancer in a subject comprising administering to the subject an effective amount of: (i) the conjugate of any one of claims 1 to 26; (ii) the nucleic acid of claim 27; (iii) the composition of claim 28 or 29; or (iv) the vaccine of claim 30 or 31.

34. Use of (i) the conjugate of any one of claims 1 to 26; (ii) the nucleic acid of claim 27; (iii) the composition of claim 28 or 29; or (iv) the vaccine of claim 30 or 31, for inducing an immune response against an immunogen in a subject.

35. Use of (i) the conjugate of any one of claims 1 to 26; (ii) the nucleic acid of claim 27; (iii) the composition of claim 28 or 29; or (iv) the vaccine of claim 30 or 31, for the manufacture of a medicament for inducing an immune response against an immunogen in a subject.

36. Use of (i) the conjugate of any one of claims 1 to 26; (ii) the nucleic acid of claim 27; (iii) the composition of claim 28 or 29; or (iv) the vaccine of claim 30 or 31, for preventing and/or treating a microbial infection or cancer in a subject.

37. Use of (i) the conjugate of any one of claims 1 to 26; (ii) the nucleic acid of claim 27; (iii) the composition of claim 28 or 29; or (iv) the vaccine of claim 30 or 31, for the manufacture of a medicament for preventing and/or treating a microbial infection, an autoimmune disease, an allergy or cancer in a subject.

38. An agent for use in inducing an immune response against an immunogen in a subject, wherein the agent is (i) the conjugate of any one of claims 1 to 26; (ii) the nucleic acid of claim 27; (iii) the composition of claim 28 or 29; or (iv) the vaccine of claim 30 or 31.

39. An agent for use in preventing and/or treating a microbial infection, an autoimmune disease, an allergy or cancer in a subject, wherein the agent is (i) the conjugate of any one of claims 1 to 26; (ii) the nucleic acid of claim 27; (iii) the composition of claim 28 or 29; or (iv) the vaccine of claim 30 or 31.

40. The (i) the conjugate of any one of claims 1 to 26; (ii) the nucleic acid of claim 27; (iii) the composition of claim 28 or 29; or (iv) the vaccine of claim 30 or 31, for use as a medicament.

41. A method for improving or increasing the immunogenicity of an immunogen comprising conjugating the immunogen to the self-assembling polypeptide defined in any one of claims 1 to 26.


SEQUENCE LISTING (29 sequences)

SEQ ID NO: 1 - protein, 41 aa, Escherichia coli
SEMTVKQFGGGNGAAVDQTASNSSVNVTQVGFGNNATAHQY

SEQ ID NO: 2 - protein, 23 aa, Influenza virus
SLLTEVETPIRNEWGSRSNGSSD

SEQ ID NO: 3 - protein, 23 aa, Escherichia coli
SELNIYQYGGGNSALALQTDARN

SEQ ID NO: 4 - protein, 22 aa, Escherichia coli
SDLTITQHGGGNGADVGQGSDD

SEQ ID NO: 5 - protein, 23 aa, Escherichia coli
SSIDLTQRGFGNSATLDQWNGKN

SEQ ID NO: 6 - protein, 41 aa, Escherichia coli
STMTVKQFGGGNGAAVDQTASNSSVNVTQVGFGNNATAHQY

SEQ ID NO: 7 - protein, 41 aa, Salmonella enterica
SDITVGQYGGNNAALVNQTASDSSVMVRQVGFGNNATANQY

SEQ ID NO: 8 - protein, 41 aa, Enterobacter ludwigii
SVMNVSQYGGGNDAAVDQTASGSTVTVQQVGFGNHATAHQY

SEQ ID NO: 9 - protein, 41 aa, Citrobacter rodentium
SDISVSQYGGNNAALVNQTASDSSVLVSQVGFGNNATANQY

SEQ ID NO: 10 - protein, 41 aa, Pseudomonas tritici
STMTVKQFGGGNGAAVDQTASGSTVSVTQVGFGNNATAHQY

SEQ ID NO: 11 - protein, 41 aa, Enterobacteriaceae bacterium
  source 1..41: note = strain ENNIH1
STISVSQYGGGNGALVNQTASGSTVNVHQVGFGNNATANQY

SEQ ID NO: 12 - protein, 41 aa, Citrobacter koseri
SDIVVSQFGGRNGALVNQTASDSQVSVTQVGFGNNATANQY

SEQ ID NO: 13 - protein, 41 aa, Trabulsiella guamensis
SEVEVRQFGASNGALVNQSASDSSVTVHQVGFGNNATASQY

SEQ ID NO: 14 - protein, 41 aa, Pseudescherichia vulneris
SDIDVRQYGTANGALVNQTASQSYVEVRQVGYGNNATANQY

SEQ ID NO: 15 - protein, 41 aa, Citrobacter tructae
ADISVTQYGGRNGAVVNQTASDSNVLVRQVGFGNNATANQH

SEQ ID NO: 16 - protein, 41 aa, Pseudescherichia vulneris; identical to SEQ ID NO: 14

SEQ ID NO: 17 - protein, 41 aa, Beauveria bassiana
AQIDVQQFGTNNGAVVNQTASSSLVSVTQFGNGNHATASQY

SEQ ID NO: 18 - protein, 41 aa, Shigella flexneri
SEMTVKQFGGGNGATVDQTASNSSVNVTQVGFGNNATAHQY

SEQ ID NO: 19 - protein, 41 aa, Shigella flexneri
SEMTVKQFGGGNGAAVDQTASNSSVNVAQVGFGNNATAHQY

SEQ ID NO: 20 - protein, 41 aa, Klebsiella pneumoniae
SEMAVKQFGGGNGAAVDQTASNSSVNVTQVGFGNNATAHQY

SEQ ID NO: 21 - protein, 41 aa, Shigella dysenteriae
SHMTVKQFGGGNGAAVDQTAFNSTVNVTQVGFGNNATAHQY

SEQ ID NO: 22 - protein, 41 aa, Lelliottia amnigena; identical to SEQ ID NO: 10

SEQ ID NO: 23 - protein, 41 aa, Pseudenterobacter timonensis
STMIVKQYGGGNGAAVDQTASGSTVSVTQVGFGNNATAHQY

SEQ ID NO: 24 - protein, 41 aa, synthetic construct
  VAR_SEQ 1: note = X is S or A
  VAR_SEQ 2: note = X is E, D, T, or V
  VAR_SEQ 3: note = X is M, I, or V
  VAR_SEQ 4: note = X is T, N, S, V, E, or D
  VAR_SEQ 6: note = X is K, G, S, R, T, or Q
  VAR_SEQ 8: note = X is an aromatic residue
  VAR_SEQ 10: note = X is G, A, or T
  VAR_SEQ 11: note = X is G, N, R, S, or A
  VAR_SEQ 13: note = X is G, A, or D
  VAR_SEQ 15: note = X is A, L, or V
  VAR_SEQ 17: note = X is D or N
  VAR_SEQ 19: note = X is S or T
  VAR_SEQ 21: note = X is S or F
  VAR_SEQ 22: note = X is N, D, G, Q, or S
  VAR_SEQ 24: note = X is S, T, Q, Y, N or L
  VAR_SEQ 26: note = X is N, M, T, L, S, or E
  VAR_SEQ 28: note = X is T, R, Q, S, H
  VAR_SEQ 30: note = X is V or F
  VAR_SEQ 32: note = X is F, Y or N
  VAR_SEQ 35: note = X is N or H
  VAR_SEQ 39: note = X is H, N, or S
  VAR_SEQ 41: note = X is Y or H
XXXXVXQXGXXNXAXVXQXAXXSXVXVXQXGXGNXATAXQX

SEQ ID NO: 25 - protein, 8 aa, synthetic construct
GGGSGGGS

SEQ ID NO: 26 - protein, 4 aa, synthetic construct
GGGS

SEQ ID NO: 27 - protein, 73 aa, Influenza virus
SLLTEVETPIRNEWGSRSNGSSDGSSLLTEVETPIRNEWGSRSNGSSDGSSLLTEVETPIRNEWGSRSNGSSD

SEQ ID NO: 28 - protein, 22 aa, Escherichia coli
SEMTVKQFGGGNGAAVDQTASN

SEQ ID NO: 29 - protein, 19 aa, Escherichia coli
SSVNVTQVGFGNNATAHQY